(19) United States
(12) Patent Application Publication
(10) Pub. No.: US 2010/0331384 A1
(43) Pub. Date: Dec. 30, 2010

(54) GUANIDINE BASED COMPOUNDS

(75) Inventors: Maria Isabel Rozas Hernando, Dublin (IE); Fernando Rodriguez Royo, La Rioja (ES); Javier Meana, Bizkaia (ES); Luis Callado, Bizkaia (ES)

Correspondence Address: SCULLY SCOTT MURPHY & PRESSER, PC, 400 GARDEN CITY PLAZA, SUITE 300, GARDEN CITY, NY, 11530, US

(73) Assignees: UNIVERSIDAD DEL PAIS VASCO/EUSKAL HERRIKO UNIBERTSITATEA, BIZKALA (ES); THE PROVOST, FELLOWS AND SCHOLARS OF THE COLLEGE OF THE HOLY AND; UNIDIVIDED TRINITY OF QUEEN ELIZABETH, DUBLIN (IE)

(21) Appl. No.: 12/809,843

(22) PCT Filed: Dec. 22, 2008
(86) PCT No.: PCT/EP08/68199

§ 371 Date: Sep. 8, 2010

(30) Foreign Application Priority Data
Dec. 21, 2007 (IE) 2007/0935

(51) Int. Cl. A61K 31/4178; C07D 233/50; C07C 279/18; C07D 403/12; A61K 31/4168; A61K 31/155; A61P 25/24; A61P 25/18; A61P 9/12; A61P 25/00; A61P 27/06

(52) U.S. Cl. 514/402; 548/333.1; 564/236; 548/314.4; 564/238; 514/401; 514/634

(57) ABSTRACT

The adrenergic receptors or adrenoceptors are a family of G-protein coupled receptors split into α and β subclasses. The adrenoceptors have important roles in regulating a myriad of physiological conditions and their malfunction has been implicated in the pathophysiology of a number of diseases. Disclosed herein are a series of novel guanidine and 2-aminoimidazoline compounds which are ligands of the alpha2-adrenoceptor (α2-ARs) subclass of adrenergic receptors. The invention also provides for pharmaceutical compositions comprising the novel compounds. The compounds are suitable for use in the manufacture of medicaments for the treatment of α2-ARs associated disorders, such as depression, schizophrenia, glaucoma and analgesia.

### FIELD OF THE INVENTION

The invention relates to compounds which are agonists and antagonists of the alpha2-adrenoceptor (α₂-ARs) subclass of adrenergic receptors. The compounds are suitable for use in the manufacture of medicaments for the treatment of alpha2-adrenoceptor (α₂-ARs) associated disorders, such as depression, schizophrenia, glaucoma and analgesia.

### BACKGROUND TO THE INVENTION

The adrenergic receptors or adrenoceptors are a family of G-protein coupled receptors split into α and β subclasses. The adrenoceptors have important roles in regulating a myriad of physiological conditions and their malfunction has been implicated in the pathophysiology of a number of diseases.

α-Adrenoceptors are further subdivided into α₁ and α₂ subclasses. The α₂ subclass of adrenoceptor can be found presynaptically, for example at nerve terminals, and postsynaptically, for example in vascular smooth muscle. Activation of presynaptic α₂ adrenoceptors inhibits noradrenaline release. Thus, antagonism of these receptors can be utilised to increase local concentrations of noradrenaline in nerve terminals.

α₂-ARs agonists have been reported as being effective in the promotion of analgesia and sedation. Furthermore, agonists of α₂-ARs have been implicated in the treatment of conditions such as hypertension and glaucoma (by decreasing intraocular pressure).

Depression is a common mental disorder that presents with depressed mood, loss of interest or pleasure, feelings of guilt or low self-worth, disturbed sleep or appetite, low energy, and poor concentration. This condition affects people of any age and sex and it has been predicted that, by 2020, depression will be the second largest health burden following only heart diseases.¹ Even though the pathophysiological origin of this disease continues to be unknown, the monoamine theory is the most widely accepted,² stating that depression is a result of a deficiency of brain monoamine (noradrenaline [NA] or serotonin) activity.

Vast research has been performed in the area of serotonin receptors, but investigations centred in the noradrenergic system remain less explored. In particular, it is well known that central noradrenergic transmission is regulated by inhibitory noradrenergic receptors (α₂-ARs) which are expressed on both somatodendritic areas and axon terminals. Hence, the activation of these α₂-ARs induces an inhibition of NA release in the brain, and thus, it has been proposed that depression is associated with a selective increase in the high-affinity conformation of the α₂-ARs in the human brain.³ This enhanced α₂-AR activity could be implicated in the deficit in noradrenergic transmission described in the aetiology of depression.

Thus, chronic treatment with antidepressants induces an in-vivo desensitization of the α₂-ARs regulating the local release of NA.⁴ Thus, the development of selective α₂-adrenoceptor antagonists can be considered as a new and effective therapeutical approach to the treatment of depressive disorders. It has been demonstrated that the administration of different α₂-AR antagonists both locally in the locus coeruleus or systemically increases the release of NA in the prefrontal cortex.^{5,6} Moreover, α₂-AR antagonists are also able to enhance the increase of NA induced by selective reuptake inhibitor antidepressant drugs.⁷

Some of the most recent antidepressants developed include Mianserin and Mirtazapine (FIG. 1), which show effective antidepressant activity, by blockade of α₂-ARs.⁸ The success of these drugs strongly supports that α₂-AR targeting, as pursued in the present work, is a promising approach for the development of new therapeutics to treat depression.

The present inventors have disclosed in a recent work⁹ the synthesis and pharmacological evaluation of a series of phenyl and di-phenyl substituted (bis)guanidine and (bis)2-aminoimidazoline derivatives with different heteroatoms in the para position with respect to these groups, as potential new antidepressants.

Compound 1 (FIG. 1) previously showed good affinity for α₂-AR targeting,¹⁰ and so this derivative was used as a lead compound and, thus, several derivatives were prepared obtaining two new α₂-AR antagonists (2 and 3, FIG. 1).

However, despite the fact that many of these molecules showed α₂-AR affinities within the range of the well-known antagonist Idazoxan (pKᵢ=7.29, see structure in Table 2), none of them improved that of the original lead compound 1 (pKᵢ=8.80).¹⁰

Thus, notwithstanding the state of the art there is a need for new compounds which are capable of targeting α₂-AR receptors and which show good α₂-AR affinities comparable with or preferably greater than Idazoxan and lead compounds 1.

### OBJECTS OF THE INVENTION

It is an object of the invention to provide compounds for the treatment of mental and neurological disorders such as depression and schizophrenia. A further object of the invention is provide a series of antidepressant drug compounds which are selective α2-adrenoceptor antagonists and which on administration both locally in the locus coeruleus or systemically increases the release of NA in the prefrontal cortex.

In a related object the present invention provides for a series of guanidine based compounds which are agonists and antagonists of the alpha2-adrenoceptor (α₂-ARs). Such compounds may find utility in the manufacture of medicaments for the treatment of alpha2-adrenoceptor (α₂-ARs) associated disorders. Compounds herein defined as antagonists may find utility in the manufacture of medicaments for the treatment of alpha2-adrenoceptor (α₂-ARs) associated disorders.

It is a further object of the invention to provide compounds for the treatment of alpha2-adrenoceptor (α₂-ARs) associated disorders selected from the group consisting of mental or neurological disorders. In particular, the alpha2-adrenoceptor (α₂-ARs) antagonists of the present invention may find utility in the manufacture of medicaments for the treatment of depression and schizophrenia.

It is an object of the invention to provide compounds for the treatment of mental and neurological disorders such as depression and schizophrenia. An object of the invention is to provide a series of antidepressant drug compounds which are selective α₂-adrenoceptor antagonists. A related object of the invention is to provide a series compounds which on administration both locally in the locus coeruleus or systemically increases the release of NA in the prefrontal cortex.

A related object is concerned with the provision of compounds which are α₂-AR antagonists, which are able to enhance the levels of NA in the synapse. Such an object is achieved by the provision of a number of symmetrical and non-symmetrical guanidine and 2-aminoimidazoline derivatives of the α₂-AR ligand compound 1 with alkyl substituents/linkers.

Compounds herein defined as agonists may find utility in the manufacture of medicaments for the treatment of alpha2-adrenoceptor (α₂-ARs) associated disorders. It is a further object of the invention to provide compounds for the treatment of alpha2-adrenoceptor (α₂-ARs) associated disorders selected from the group consisting of analgesia, hypertension or glaucoma. In particular, the alpha2-adrenoceptor (α₂-ARs) agonists of the present invention may find utility in the manufacture of medicaments for analgesia and the treatment of glaucoma.

A further object still is the provision of efficient synthetic methods to allow preparation of symmetrical and non-symmetrical guanidine and 2-aminoimidazoline derivatives of the α₂-AR ligand compound 1 with alkyl substituents/linkers.

A further object of the invention is to demonstrate that such compounds can be used as new and effective therapeutics or in the manufacture of such therapeutics for use in the treatment of depressive disorders.

Another object is the design and provision of pharmacological analytic methods that allow the use of human brain tissue to directly characterize the pharmacological properties of the new compounds. Characterisation of such properties is relevant from a therapeutic perspective.

### SUMMARY OF THE INVENTION

In one aspect the present invention provides for a compound, or a pharmaceutically acceptable salt thereof, comprising a guanidine core having three nitrogen atoms bonded to a central carbon atom and wherein carbon-nitrogen bonds comprise an imine functional group or amine functional groups,

wherein one of the nitrogen atoms is substituted with a fused tricylic ring comprising a fluorene ring or a dihydroanthracene ring, or a bibenzyl ring which are unsubstituted or substituted with at least one C₁-C₅ alkyl group, a guanidine group or a 4,5-dihydro-1H-imidazol-2-amine group; or

wherein one of the nitrogen atoms is substituted with a fused bicyclic ring comprising a tetrahydronapthalene ring which is unsubstituted or substituted with at least one C₁-C₅ alkyl group; and

the remaining nitrogen atoms are substituted with hydrogen or a bridging C₁-C₅ alkyl group to form a cyclic heteroatom ring.

In a further aspect, the present invention provides for a compound, or a pharmaceutically acceptable salt thereof, comprising a guanidine core having three nitrogen atoms bonded to a central carbon atom and wherein carbon-nitrogen bonds comprise an imine functional group or amine functional groups,

wherein one of the nitrogen atoms is substituted with a benzene ring which is substituted with a C₁-C₅ alkyl group or a benzene ring di-substituted with a C₁-C₅ alkyl group; and

the remaining nitrogen atoms are substituted with hydrogen or a bridging C₁-C₅ alkyl group to form a cyclic heteroatom ring.

According to the present invention there is provided a compound or a pharmaceutically acceptable salt thereof wherein the compound has the general formula

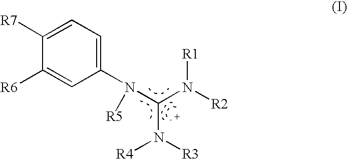

wherein the imine functional group can be at any one of the guanidine core carbon-nitrogen bonds; and

R₁ is H, N-tert-butoxycarbonate group, a lone pair of electrons or a C₁-C₅ alkyl chain which may be substituted or unsubstituted;

R₂ is H, a lone pair of electrons, a N-tert-butoxycarbonate group or a C₁-C₅ alkyl chain which may be substituted or unsubstituted;

R₃ is H, a lone pair of electrons, a N-tert-butoxycarbonate group or a C₁-C₅ alkyl chain which may be substituted or unsubstituted;

R₄ is H, N-tert-butoxycarbonate group or a lone pair of electrons or a C₁-C₅ alkyl chain which may be substituted or unsubstituted; or R₂ and R₃ together form a cyclic ring structure; and

R₅ is H, C₁-C₅ alkyl or a lone pair of electrons;

R₆ is H, an aryl, a C₁-C₅ alkyl aryl or a C₁-C₅ alkyl group, which may be substituted or unsubstituted; and

R₇ is H, an aryl, a C₁-C₅ alkyl aryl or a C₁-C₅ alkyl group, which may be substituted or unsubstituted.

R₆ and R₇ may together form part of a cyclic ring structure, a fused bicyclic or a fused tricyclic ring which can be unsubstituted or substituted, wherein the fused bicyclic ring is a diphenylmethane ring or a tetrahydronapthalene ring, which are unsubstituted or substituted with at least one of a C₁-C₅ alkyl, an aryl, or a C₁-C₅ alkyl aryl group. The tricylic ring may be a fluorene ring, a dihydroanthracene ring or a biaryl or a bialkylaryl ring, which are unsubstituted or substituted at least one of a C₁-C₅ alkyl, an aryl, a C₁-C₅ alkyl aryl group, a guanidine group or a 4,5-dihydro-1H-imidazol-2-amine group.

In yet a further aspect the present invention provides for a compound or a pharmaceutically acceptable salt thereof wherein the compound has the general formula (I)

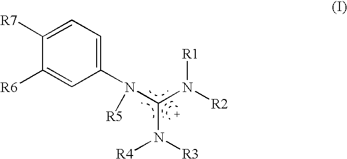

wherein the imine functional group can be at any one of the guanidine core carbon-nitrogen bonds; and

R₁ is H, N-tert-butoxycarbonate group, a lone pair of electrons or a C₁-C₅ alkyl chain which may be substituted or unsubstituted;

R₂ is H, a lone pair of electrons, a N-tert-butoxycarbonate group or a C₁-C₅ alkyl chain which may be substituted or unsubstituted;

R₃ is H, a lone pair of electrons, a N-tert-butoxycarbonate group or a C₁-C₅ alkyl chain which may be substituted or unsubstituted;

R₄ is H, N-tert-butoxycarbonate group or a lone pair of electrons or a C₁-C₅ alkyl chain which may be substituted or unsubstituted; or R₂ and R₃ together form a cyclic ring structure; and

R₅ is H, C₁-C₅ alkyl or a lone pair of electrons;

R₆ is H, an aryl, a C₁-C₁₀ alkyl aryl, phenylmethyl, 2-phenylethyl or a C₁-C₅ alkyl group, which may be substituted or unsubstituted, wherein when R₆ comprises phenylmethyl it is not substituted with a guanidine group or a 4,5-dihydro-1H-imidazol-2-amine group; and

R₇ is H, an aryl, a C₁-C₁₀ alkyl aryl, phenylmethyl, 2-phenylethyl or a C₁-C₅ alkyl group, which may be substituted or unsubstituted, with the proviso that when R₇ comprises phenylmethyl it is not substituted with a guanidine group or a 4,5-dihydro-1H-imidazol-2-amine group; or

R₆ and R₇ together form part of a cyclic ring structure, a fused bicyclic or a fused tricyclic ring which can be unsubstituted or substituted,

with the proviso that when R₆ and R₇ form part of a fused bicyclic ring, R₆ and R₇ do not comprise a dioxane ring or a dioxolane ring, and

further provided that when R₂ and R₃ together form a cyclic ring structure and when R₆ and R₇ form part of a fused bicyclic ring, R₆ and R₇ comprise an unsubstituted tetrahydronapthalene ring.

In one embodiment R₆ and/or R₇ may comprise C₁-C₅ alkyl aryl. Desirably, R₁ to R₅ are H, or R₁ and R₄ to R₅ are H and R₂ and R₃ together form a 5 membered cyclic ring structure. R₆ and/or R₇ may be phenylmethyl, 2-phenylethyl or a C₁-C₅ alkyl group, which may be substituted or unsubstituted, with the proviso that when R₆ and/or R₇ comprises phenylmethyl it is not substituted with a guanidine group or a 4,5-dihydro-1H-imidazol-2-amine group. Desirably, R₇ is phenylmethyl, 2-phenylethyl or a C₁-C₅ alkyl, wherein the 2-phenylethyl group is substituted with a 4,5-dihydro-1H-imidazol-2-amine group.

As used herein the term “substituted” refers to substitution with at least one of a halogen, oxygen, nitrogen, sulfur, C₁-C₅ alkyl, an aryl, a C₁-C₁₀ alkyl aryl group, a guanidine group or a 4,5-dihydro-1H-imidazol-2-amine group. Desirably, the term “substituted” refers to substitution with at least one of one of a C₁-C₅ alkyl, an aryl, a C₁-C₁₀ alkyl aryl group, a guanidine group or a 4,5-dihydro-1H-imidazol-2-amine group.

As used herein, the term “substituted” with reference to R₆ and R₇ refers to halogen, hydroxy, amine, thiol etc. substitution branching from the main alkyl chain. The definition of substitution does not embrace substitution of a carbon atom in the main C₁-C₅ alkyl chain with a heteroatom such as O, N, or S, e.g. ether, thioether or amine linkages.

In one embodiment, the present invention provides for a compound of the general formula (I), or a pharmaceutically acceptable salt thereof, wherein R₆ and R₇ together form part of a fused tricyclic ring. The fused tricylic ring may be selected from a fluorene ring, a dihydroanthracene ring or a bisaryl or a bisalkylaryl ring, which are unsubstituted or substituted with at least one of a C₁-C₅ alkyl, an aryl, a C₁-C₅ alkyl aryl group, a C₁-C₁₀ alkyl aryl group a guanidine group or a 4,5-dihydro-1H-imidazol-2-amine group. Desirably, the fused tricylic ring is selected from a fluorene ring, or a dihydroanthracene ring, which are unsubstituted or substituted with at least one of a 0₁-C₅ alkyl, an aryl, a C₁-C₅ alkyl aryl group, C₁-C₁₀ alkyl aryl group, a guanidine group or a 4,5-dihydro-1H-imidazol-2-amine group. When R₆ or R₇ comprise 2-phenylethyl, or R₆ and R₇ together form part of a fused tricyclic ring the resulting structures may be substituted with at least one of a guanidine group or a 4,5-dihydro-1H-imidazol-2-amine group.

In certain embodiments, the compounds of the invention is selected from the group comprising

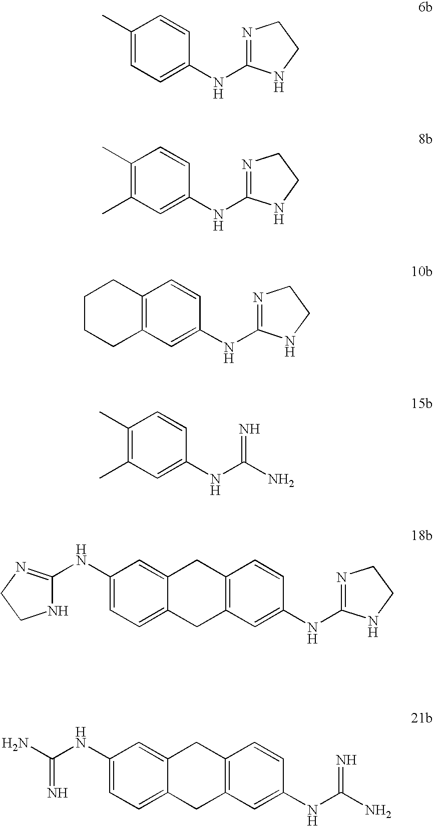

or a pharmaceutically acceptable salt thereof, as an α₂-AR agonist.

The compound according to the present invention may be selected from the group comprising

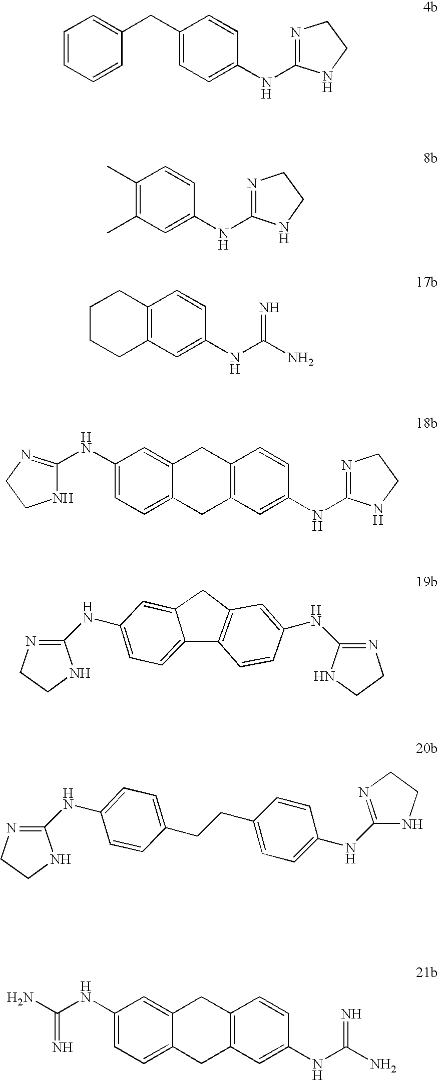

In one embodiment, where alpha2-adrenoceptor agonists are required, a compound according to the present invention can be selected from the group comprising

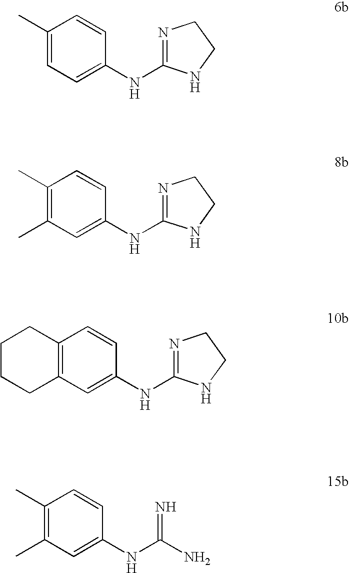

An agonist compound according to the present invention may further comprise:

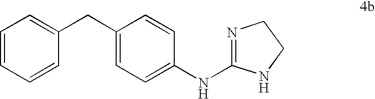

In a further embodiment, where alpha2-adrenoceptor antagonistic properties are required, a compound according to the present invention can be selected from the group comprising:

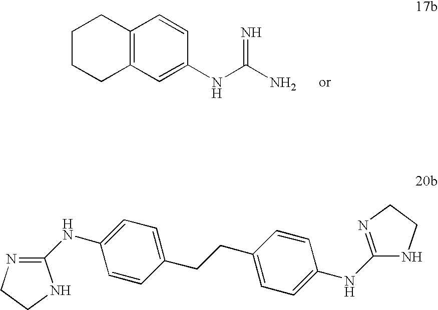

In yet a further embodiment, a compound according to the present invention may be selected from the group comprising:

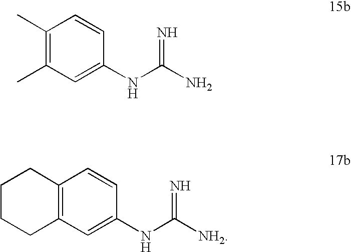

The invention further provides for a pharmaceutical composition comprising a compound according to the present invention together with a pharmaceutical acceptable carrier or excipient(s). The pharmaceutical composition may comprise an antagonist compound according to the present invention. Alternatively, the pharmaceutical composition may comprise an agonist compound according to the present invention.

According to the present invention there is further provided for use a compound or a pharmaceutically acceptable salt thereof wherein the compound has the general formula (I)

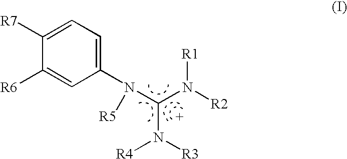

wherein the imine functional group can be at any one of the guanidine core carbon-nitrogen bonds; and

R₁ is H, N-tert-butoxycarbonate group, a lone pair of electrons or a C₁-C₅ alkyl chain which may be substituted or unsubstituted;

R₂ is H, a lone pair of electrons, a N-tert-butoxycarbonate group or a C₁-C₅ alkyl chain which may be substituted or unsubstituted;

R₃ is H, a lone pair of electrons, a N-tert-butoxycarbonate group or a C₁-C₅ alkyl chain which may be substituted or unsubstituted;

R₄ is H, N-tert-butoxycarbonate group or a lone pair of electrons or a C₁-C₅ alkyl chain which may be substituted or unsubstituted; or R₂ and R₃ together form a cyclic ring structure; and

R₅ is H, C₁-C₅ alkyl or a lone pair of electrons;

R₆ is H, an aryl, a C₁-C₅ alkyl aryl or a C₁-C₅ alkyl group, which may be substituted or unsubstituted; and

R₇ is H, an aryl, a C₁-C₅ alkyl aryl or a C₁-C₅ alkyl group, which may be substituted or unsubstituted,

in the manufacture of a medicament for the treatment of alpha2-adrenoceptor (α₂-ARs) associated disorders.

R₆ and R₇ may together form part of a cyclic ring structure, a fused bicyclic or a fused tricyclic ring which can be unsubstituted or substituted, wherein the fused bicyclic ring is a diphenylmethane ring or a tetrahydronapthalene ring, which are unsubstituted or substituted with at least one of a C₁-C₅ alkyl, an aryl, or a C₁-C₅ alkyl aryl group.

The tricylic ring may be a fluorene ring, a dihydroanthracene ring or a biaryl or a bialkylaryl ring, which are unsubstituted or substituted at least one of a C₁-C₅ alkyl, an aryl, a C₁-C₅ alkyl aryl group, a guanidine group or a 4,5-dihydro-1H-imidazol-2-amine group.

In yet a further aspect the present invention provides for use of a compound of the general formula (I) or a pharmaceutically acceptable salt thereof,

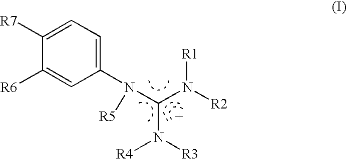

wherein the imine functional group can be at any one of the guanidine core carbon-nitrogen bonds; and

R₁ is H, N-tert-butoxycarbonate group, a lone pair of electrons or a C₁-C₅ alkyl chain which may be substituted or unsubstituted;

R₂ is H, a lone pair of electrons, a N-tert-butoxycarbonate group or a C₁-C₅ alkyl chain which may be substituted or unsubstituted;

R₃ is H, a lone pair of electrons, a N-tert-butoxycarbonate group or a C₁-C₅ alkyl chain which may be substituted or unsubstituted;

R₄ is H, N-tert-butoxycarbonate group or a lone pair of electrons or a C₁-C₅ alkyl chain which may be substituted or unsubstituted; or R₂ and R₃ together form a cyclic ring structure; and

R₅ is H, C₁-C₅ alkyl or a lone pair of electrons;

R₆ is H, an aryl, a C₁-C₅ alkyl aryl, phenylmethyl, 2-phenylethyl or a C₁-C₅ alkyl group, which may be substituted or unsubstituted, wherein when R₆ comprises phenylmethyl it is not substituted with a guanidine group or a 4,5-dihydro-1H-imidazol-2-amine group; and

R₇ is H, an aryl, a C₁-C₅ alkyl aryl, phenylmethyl, 2-phenylethyl or a C₁-C₅ alkyl group, which may be substituted or unsubstituted, with the proviso that when R₇ comprises phenylmethyl it is not substituted with a guanidine group or a 4,5-dihydro-1H-imidazol-2-amine group; or

R₆ and R₇ together form part of a cyclic ring structure, a fused bicyclic or a fused tricyclic ring which can be unsubstituted or substituted,

with the proviso that when R₆ and R₇ form part of a fused bicyclic ring, R₆ and R₇ do not comprise a dioxane ring or a dioxolane ring, and

further provided that when R₂ and R₃ together form a cyclic ring structure and when R₆ and R₇ form part of a fused bicyclic ring, R₆ and R₇ comprise an unsubstituted tetrahydronapthalene ring,

in the manufacture of a medicament for the treatment of alpha2-adrenoceptor (α₂-ARs) associated disorders.

In one embodiment R₆ and/or R₇ may comprise C₁-C₅ alkyl aryl. Desirably, R₁ to R₅ are H, or R₁ and R₄ to R₅ are H and R₂ and R₃ together form a 5 membered cyclic ring structure. R₆ and/or R₇ may be phenylmethyl, 2-phenylethyl or a C₁-C₅ alkyl group, which may be substituted or unsubstituted, with the proviso that when R₆ and/or R₇ comprises phenylmethyl it is not substituted with a guanidine group or a 4,5-dihydro-1H-imidazol-2-amine group. Desirably, R₇ is phenylmethyl, 2-phenylethyl or a C₁-C₅ alkyl, wherein the 2-phenylethyl group is substituted with a 4,5-dihydro-1H-imidazol-2-amine group.

Use according to the present invention may comprise use of a compound of the general formula (I), or a pharmaceutically acceptable salt thereof, wherein R₆ and R₇ together form part of a fused tricyclic ring. The fused tricylic ring may be selected from a fluorene ring, a dihydroanthracene ring or a bisaryl or a bisalkylaryl ring, which are unsubstituted or substituted with at least one of a C₁-C₅ alkyl, an aryl, a C₁-C₅ alkyl aryl group, a guanidine group or a 4,5-dihydro-1H-imidazol-2-amine group. Desirably, the fused tricylic ring is selected from a fluorene ring, or a dihydroanthracene ring, which are unsubstituted or substituted with at least one of a C₁-C₅ alkyl, an aryl, a C₁-C₅ alkyl aryl group, a guanidine group or a 4,5-dihydro-1H-imidazol-2-amine group. When R₆ or R₇ comprise 2-phenylethyl, or R₆ and R₇ together form part of a fused tricyclic ring the resulting structures may be substituted with at least one of a guanidine group or a 4,5-dihydro-1H-imidazol-2-amine group.

Use according to the present invention may comprise a compound of the general formula (I), or a pharmaceutically acceptable salt thereof, which can be selected from the group consisting of:

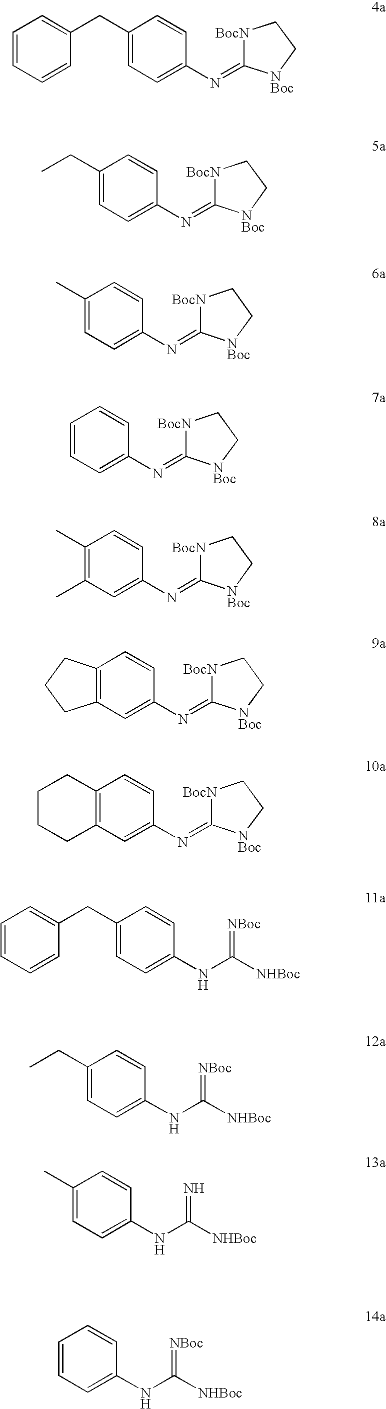

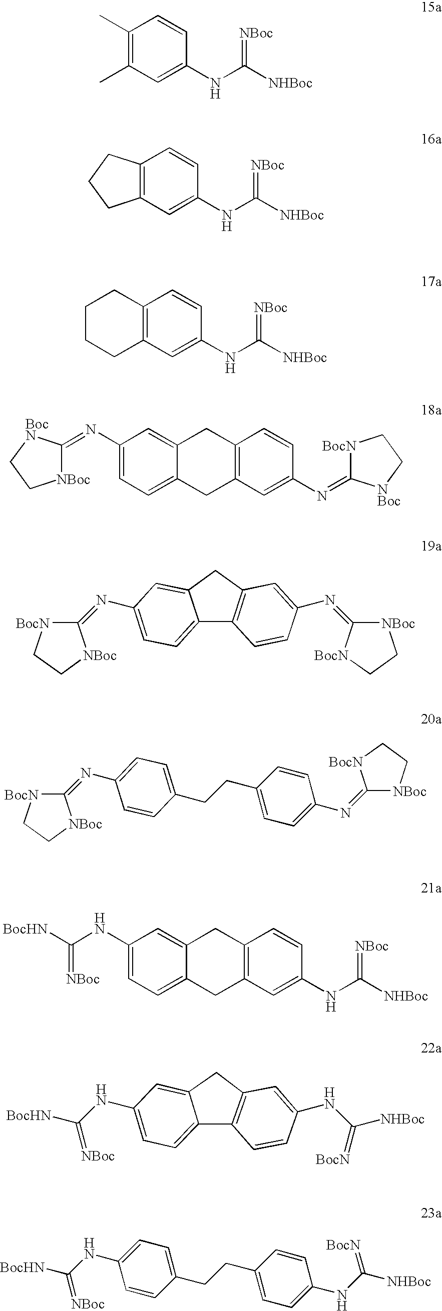

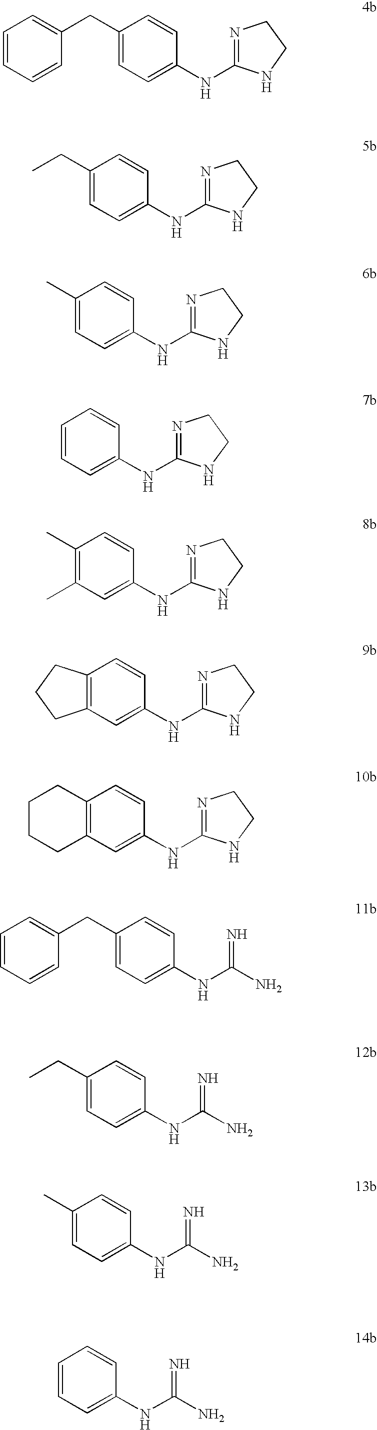

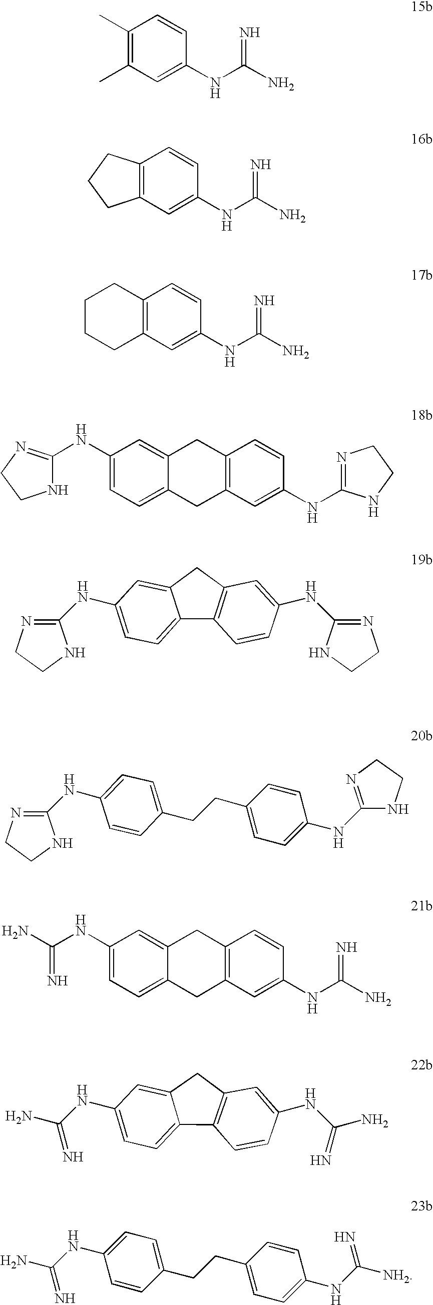

Use according to the present invention may further comprise compounds of the general formula (I), or a pharmaceutically acceptable salt thereof, selected from the group comprising:

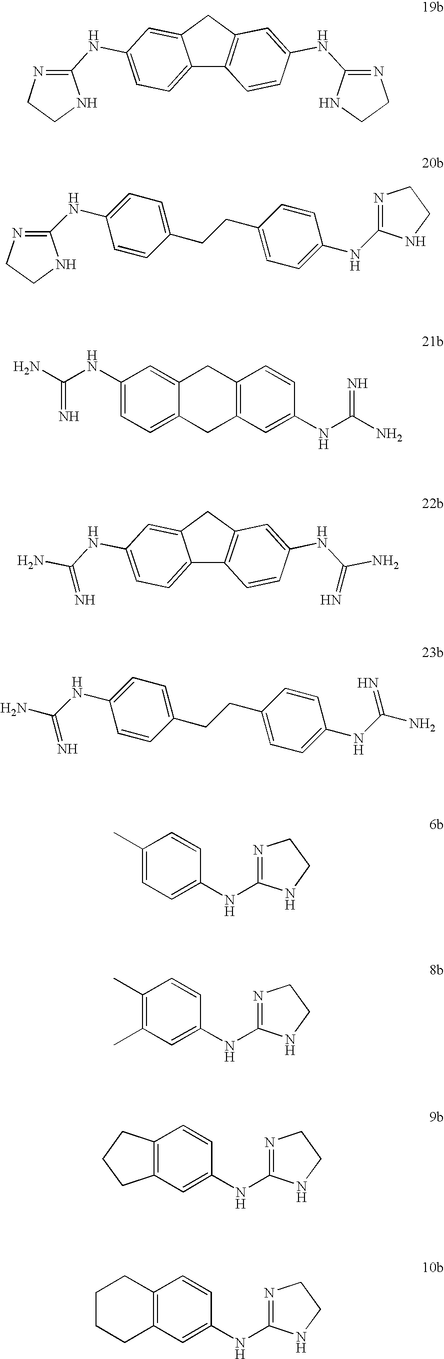

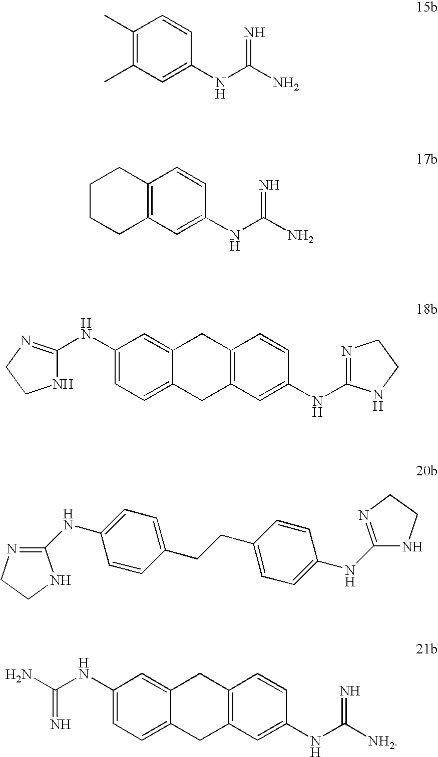

In an alternative embodiment, use according to the present invention may further comprise compounds of the general formula (I), or a pharmaceutically acceptable salt thereof, selected from the group comprising symmetric compounds

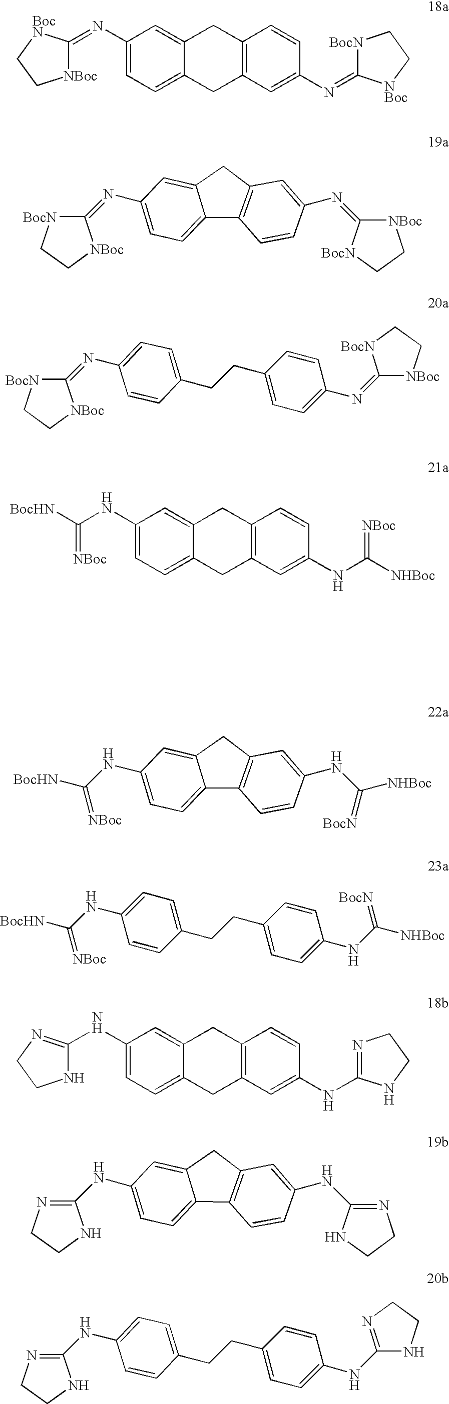

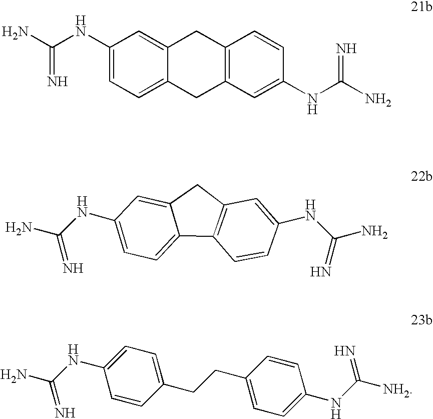

Use according to the present invention may yet further comprise compounds of the general formula (I), or a pharmaceutically acceptable salt thereof, selected from the group comprising

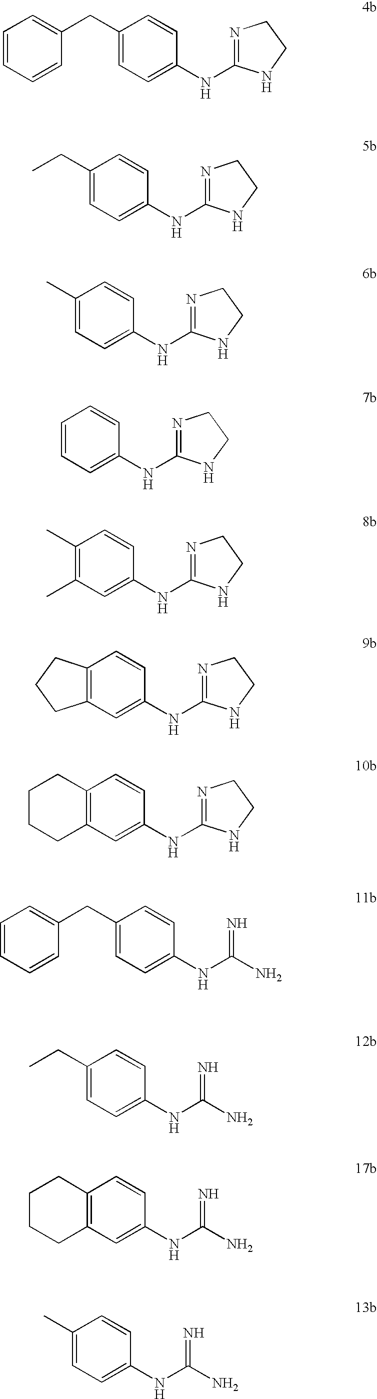

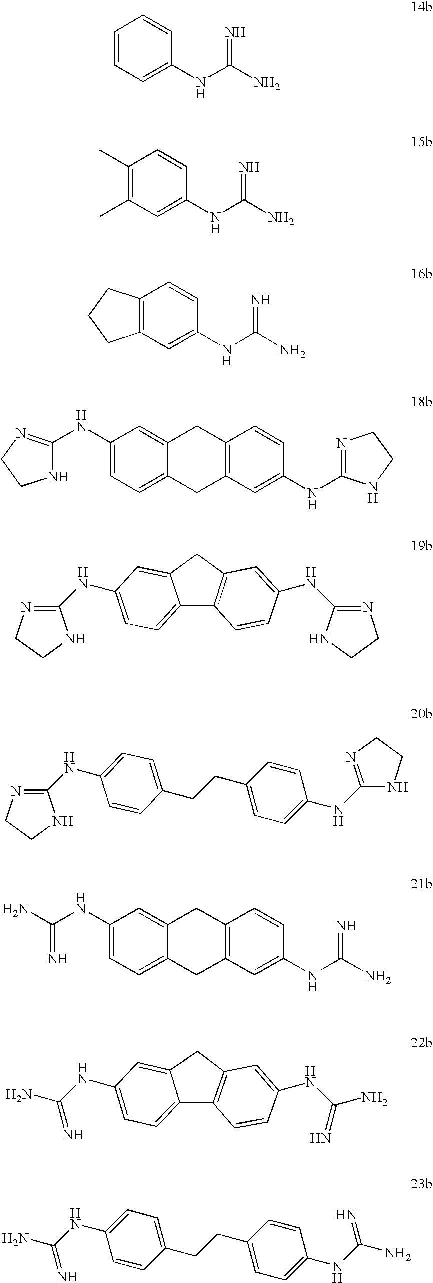

Use according to the present invention for guanidine compounds of the general formula (I), or a pharmaceutically acceptable salt thereof, may be selected from the group comprising

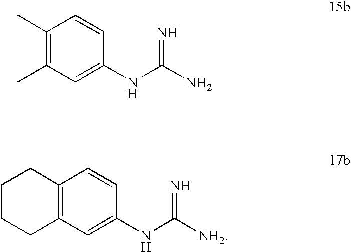

In a further embodiment, use according to the present invention comprises an alpha2-adrenoceptor (α₂-ARs) agonist compound of the general formula (I), or a pharmaceutically acceptable salt thereof, selected from the group comprising:

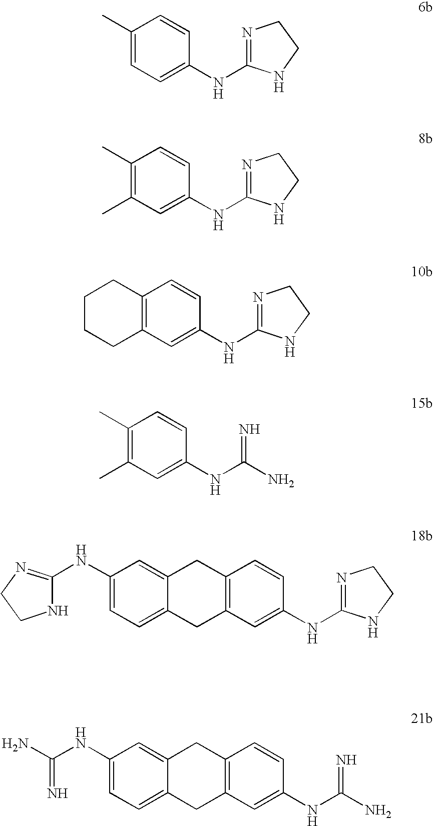

In one embodiment, use according to the present invention comprises an alpha2-adrenoceptor (α₂-ARs) agonist compound of the general formula (I), or a pharmaceutically acceptable salt thereof, selected from the group comprising

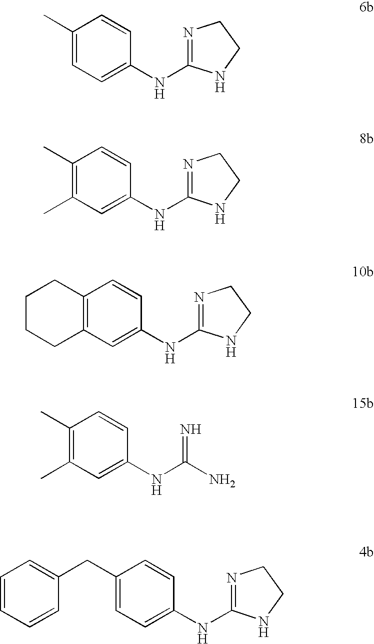

Desirably, use according to the present invention comprises an α₂-AR agonist of the general formula (I), or a pharmaceutically acceptable salt thereof, selected from the group consisting of

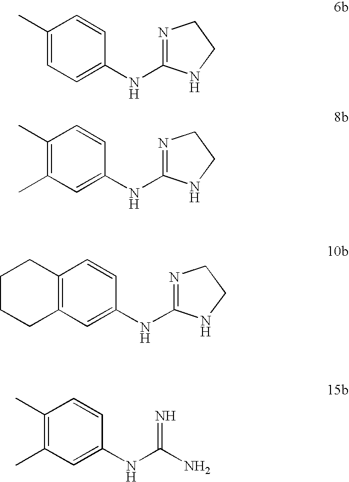

Use according to the present invention may further comprise α₂-AR agonist compounds of the general formula (I), or a pharmaceutically acceptable salt thereof, selected from the group comprising:

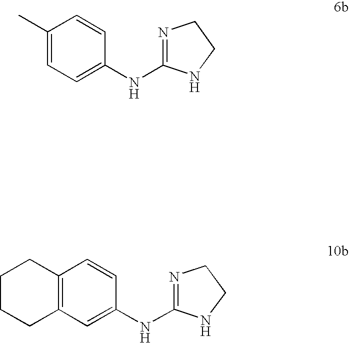

The alpha2-adrenoceptor (α₂-ARs) agonists of the present invention may find utility in the manufacture of medicaments for analgesia or for the treatment of at least one of hypertension or glaucoma. Further desirably, the alpha2-adrenoceptor (α₂-ARs) agonists of the present invention may find utility in the manufacture of medicaments for analgesia and the treatment of glaucoma.

Use according to the present invention may further comprise antagonist compounds of the general formula (I), or a pharmaceutically acceptable salt thereof, selected from the group comprising

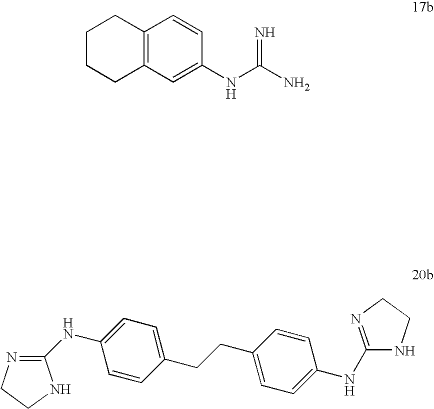

The alpha2-adrenoceptor (α₂-ARs) antagonists of the present invention may find utility in the manufacture of medicaments for the treatment of least one of mental or neurological disorders. Desirably, mental or neurological disorders comprise at least one of depression or schizophrenia. Further desirably, the alpha2-adrenoceptor (α₂-ARs) antagonists of the present invention may find utility in the manufacture of medicaments for the treatment of depression.

The invention further provides for a method of treating an alpha2-adrenoceptor associated disorder in a patient in need thereof, comprising administering to the patient a pharmaceutically effective amount of a compound according to the present invention or a pharmaceutically acceptable salt thereof.

In one embodiment of the method of treatment of the present invention, the compound is selected from the group comprising:

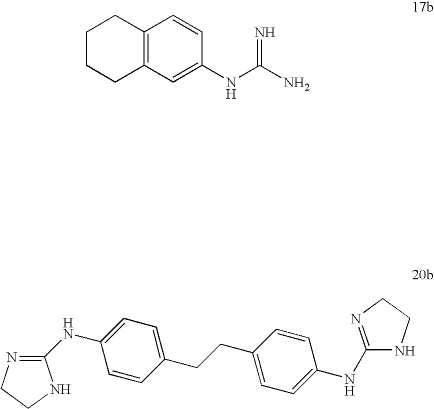

or a pharmaceutically acceptable salt thereof, and the alpha2-adrenoceptor associated disorder is selected from at least one of depression or schizophrenia.

In a further embodiment of the method of treatment of the present invention, the compound is selected from the group comprising:

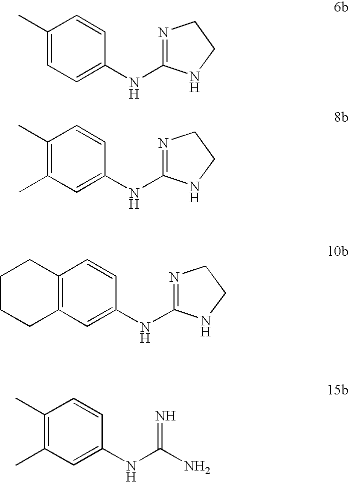

or a pharmaceutically acceptable salt thereof, and the alpha2-adrenoceptor associated disorder is selected from at least one of analgesia, hypertension or glaucoma.

In a further aspect the invention extends to a compound, or a pharmaceutically acceptable salt thereof, substantially as described herein and with reference to the accompanying examples; a pharmaceutical composition substantially as described herein and with reference to the accompanying examples; and use of compound substantially as described herein and with reference to the accompanying examples. In conclusion, a series of compounds were prepared which show α₂-AR affinities comparable with or greater than the range of the well-known antagonist Idazoxan (pKᵢ=7.29, see structure in Table 2) and further improved over that of the original lead compound 1 (pKᵢ=8.80).¹⁰

Compounds with excellent α₂-AR affinities were designed using lead compound 1 as a basis but avoiding the presence of heteroatoms in the linker. Different mono- and dicationic analogues of 1 were pharmacologically tested keeping the methylene (or other alkyl groups) in the linker position in order to establish some Structure Activity Relationships (SARs).

Thus the invention provides not only the efficient synthesis of a number of symmetrical and non-symmetrical guanidine and 2-aminoimidazoline derivates of the α₂-AR ligand compound 1 with alkyl substituents/linkers, but also, and more importantly, a complete pharmacological study of the affinity of these compounds and antagonistic activity in human brain tissue, and their in vivo antagonistic activity in rats.

In vitro assays in human brain tissue to evaluate the α₂-AR affinity and functional studies to determine the agonist or antagonist nature of those derivatives with pKᵢ>7 were designed and performed. Generally, such tests are carried out in animal tissue. Furthermore, in vivo microdialysis experiments in rats were carried out with the compounds showing antagonistic properties, to test their effect on NA release in order to establish their potential use as antidepressants. As mentioned, the α₂-AR affinity and potential receptor antagonism experiments were performed in human prefrontal cortex (PFC), since there is an important density of α₂-ARs in this tissue.” Moreover, many studies have reported changes in PFC activity in the brain of patients with depression.¹²

Herein is reported the quick and efficient synthesis of a number of (bis)guanidine and (bis)2-aminoimidazoline derivatives. The final compounds 6b, 8b, 9b, 10b, 15b, 17b, 18b, 20b and 21b showed affinities towards the α₂-ARs in human brain tissue in in vitro experiments within the range of those of Idazoxan and/or Clonidine. Compounds 18b, 20b and 21b are twin molecules, whereas 6b, 8b, 9b, 10b, 15b and 17b are monocationic derivatives. Compounds 6b, 8b, 9b, 10b, 18b and 20b are 2-aminoimidazoline derivatives, whilst 15b, 17b and 21b are guanidine containing substrates. Generally speaking, the 2-aminoimidazoline derivatives displayed higher affinities towards the α₂-ARs than their guanidine analogues, as expected from the results obtained in our first work.⁹ However, remarkably, since previous antagonists had been monocations and not twin (symmetrical) dications, and for the first time a guanidine twin compound (21 b) with a pKᵢ>7 was obtained.

In terms of activity, compounds 6b, 8b, 9b, 10b, 15b, 18b, and 21b showed agonistic properties in the [³⁵S]GTPγS experiments carried out. It is not an obvious task to explain the different behaviour found for 9b and the dioxo compound 3⁹ in the α₂-ARs, since they share basically the same backbone except for the presence of the two oxygen atoms in the latter one, which could be establishing some relevant interactions for the antagonism.

An important result achieved in this work is the identification of compounds 17b and 20b, which displayed antagonistic properties both in vitro [³⁵S]GTPγs binding experiments and in vivo microdialysis experiments. Remarkably, 17b is the first guanidine containing derivative prepared by the inventors with such characteristics. Yet again, very subtle structural changes led to different activity in the α₂-ARs. Comparing the structures of 17b and 31b⁹ one might expect similar behaviour from both, however, the former one turned out to be an antagonist whilst the dioxo compound 31b⁹ is an agonist. This is the antithesis for the 2-aminoimidazoline derivatives 9b and 3 (supra).⁹ As a result, no obvious SAR can be formulated with regard to the observed activities.

Considering the results of this series of in vivo microdialysis experiments it can be concluded that the antagonistic properties of compounds 17b and 20b over α₂-ARs, as expected from their behaviour in [³⁵S]GTPγs binding experiments, are confirmed.

Where suitable, it will be appreciated that all optional and/or preferred features of one embodiment of the invention may be combined with optional and/or preferred features of another/other embodiment(s) of the invention.

### BRIEF DESCRIPTION OF THE DRAWINGS

Additional features and advantages of the present invention are described in, and will be apparent from, the detailed description of the invention and from the drawings in which:

FIG. 1 illustrates structures of known α₂-noradrenoceptor targeting antidepressants and those of the high α₂-AR affinity compound 1 and two α₂-AR antagonists (2 and 3) previously described;

FIG. 2 illustrates the effects of local administration (1-100 μM) by reverse microdialysis in the PFC of 17b, 20b, Idazoxan, RX821002 or cerebrospinal fluid. Concentration of the compounds were progressively increased every two fractions (70 min) in tenfold increments (arrows). Data are given as mean±standard error mean values from 3 to 4 separate animals for each group, and are expressed as percentages of the corresponding basal values.

FIG. 3 illustrates the effects of the systemic administration of 17b, 20b or saline on extracellular NA levels, evaluated in the PFC. Data are given as mean±standard error mean values from 3 separate animals for each group and are expressed as percentages of the corresponding basal values. Arrow represents administration of the different compounds.

FIG. 4 illustrates the tail suspension test results for compounds of the present invention as a method for assessing antidepressant-like activity in mice.

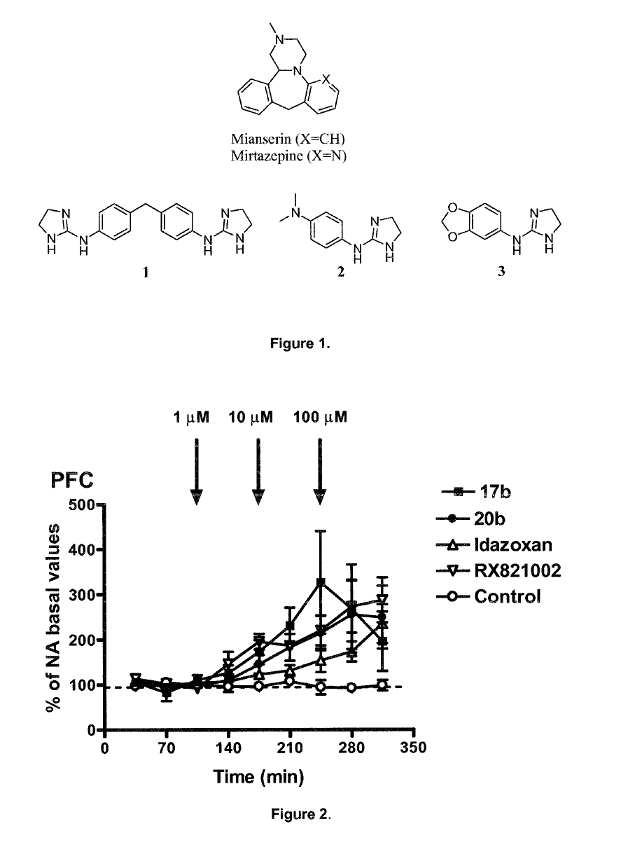

### DETAILED DESCRIPTION OF THE INVENTION

### Chemistry

The importance of the roles played by different guanidine and 2-aminoimidazoline containing compounds in biological processes has led to the development of various methodologies that allow introduction of both these groups into the backbone of different molecules.⁹ Amongst them, based on the features of our starting materials and the good results obtained previously,^{9,13} we decided to use Kim and Qian's strategy.¹⁴ This approach consists of the treatment of the corresponding starting amine (or diamine) with one (or two) equivalent(s) of either N,N′-bis(tert-butoxycarbonyl)thiourea or N,N′-di(tert-butoxycarbonyl)imidazoline-2-thione¹³ (as the guanidine and 2-aminoimidazoline precursors respectively) in the presence of mercury (II) chloride and an excess of triethylamine (Scheme 1).

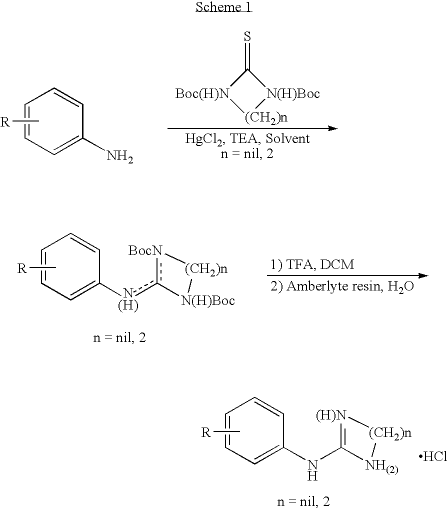

The Boc-protected guanidine intermediates obtained in the first step of the synthesis were purified by silica gel column chromatography, whereas in the case of 2-aminoimidazoline precursor ones, a quick flash column chromatography over neutral alumina was run instead. Afterwards, some of the substrates required to be recrystallised from the appropriate solvent.

TABLE 1
Overall, first and second stage yields (in %) obtained for all compounds
prepared.

| | | 1^{st} | | | 2^{nd} | Over- |
|---|---|---|---|---|---|---|
| Compd | Structure | Stage | Compd | Structure | Stage | all |
| 4a | 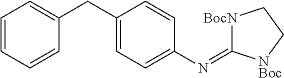 | 78 | 4b | 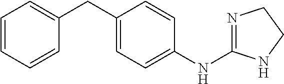 | 96 | 75 |
| 5a | 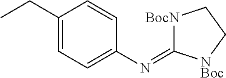 | 80 | 5b | 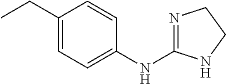 | 95 | 76 |
| 6a | 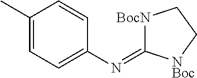 | 69 | 6b | 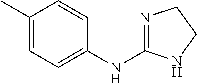 | 94 | 65 |
| 7a | 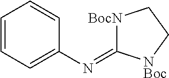 | 82 | 7b | 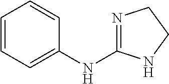 | 97 | 80 |
| 8a | 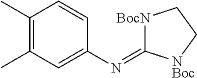 | 62 | 8b | 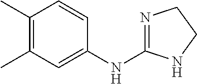 | 96 | 60 |
| 9a | 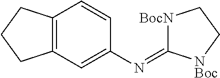 | 65 | 9b | 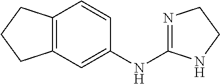 | 95 | 62 |
| 10a | 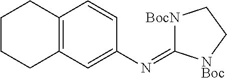 | 79 | 10b | 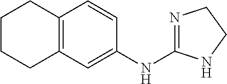 | 94 | 74 |
| 11a | 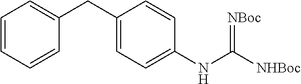 | 81 | 11b | 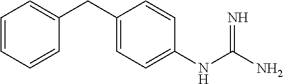 | 97 | 78 |
| 12a | 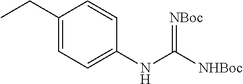 | 77 | 12b | 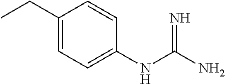 | 94 | 72 |
| 13a | 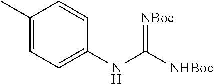 | 81 | 13b | 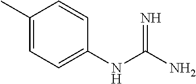 | 98 | 79 |
| 14a | 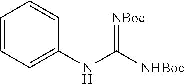 | 73 | 14b | 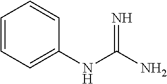 | 96 | 70 |
| 15a | 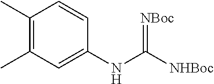 | 86 | 15b | 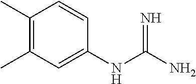 | 97 | 83 |
| 16a | 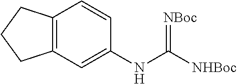 | 81 | 16b | 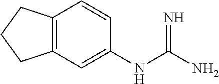 | 96 | 78 |
| 17a | 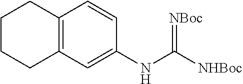 | 78 | 17b | 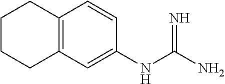 | 95 | 74 |
| 18a | 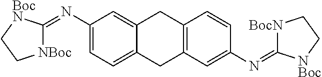 | 70 | 18b | 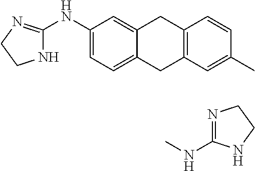 | 94 | 66 |
| 19a | 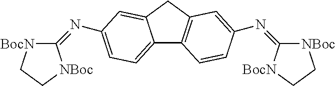 | 71 | 19b | 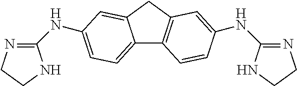 | 95 | 67 |
| 20a | 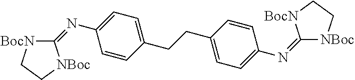 | 71 | 20b | 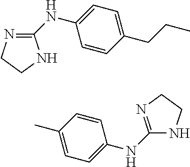 | 96 | 68 |
| 21a | 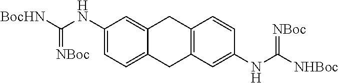 | 61 | 21b | 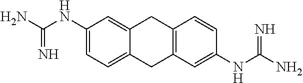 | 95 | 58 |
| 22a | 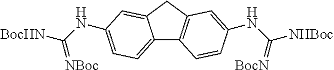 | 81 | 22b | 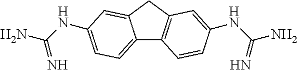 | 95 | 77 |
| 23a | 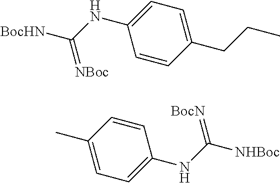 | 62 | 23b | 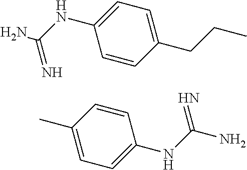 | 94 | 58 |

Standard Boc deprotection of the intermediates with an excess of trifluoroacetic acid in dichloromethane and further treatment with Amberlyte resin in aqueous solution led to the hydrochloride salts of the target molecules in good overall yields (Table 1). All the starting amines are commercially available either from Aldrich or Fluka, except for the 9,10-dihydroanthracene-2,6-diamine, which was synthesised according to the procedure reported in the literature.¹⁵

The Boc-protected derivatives 14a¹⁶ and 22a¹⁷ have been previously described; however, none of them was prepared by Kim and Qian's methodology. Regarding the final substrates, compounds 4b, 8b, 10b, 17b, 18b, 19b, 20b and 21b are new, whilst 5b, 6b, 7b, 11b, 12b, 13b, 14b, 15b, 16b, 22b, and 23b, although previously described as synthetic intermediates or prepared for other purposes, have not been tested in human brain tissue as possible antidepressants. It is also important to mention that compound 9b¹⁸ is disclosed in International Patent Application WO9846572 and reported as a cloned human α₂-AR receptor agonist, but, since it had not been tested in human brain PFC, we included it in our study for the sake of comparison. All the compounds have been tested in their hydrochloride salt form.

### Pharmacology

The affinity towards the α₂-ARs in human brain PFC tissue of all compounds prepared was measured by competition with the selective radioligand [³H]RX821002 (2-methoxy-idazoxan), which was used at a constant concentration of 1 nM.

### Affinity of the Monocationic Compounds

Design of the new compounds was based on fragments of the structure of the original lead compound 1. This would allow us not only to understand the importance of the presence of the second cation in the affinity towards the α₂-ARs and its activity, but also to explore the effect of keeping the methylene group in the linker instead of the heteroatomic —O—, —S— and —NH— groups in the related analogues previously reported.⁹ In addition, the guanidine containing substrates were subject to study because of the similarities previously found between the guanidinium and the 2-aminoimidazolinium cations.¹⁹

The affinities towards the α₂-ARs (expressed as pKᵢ) of all the monocationic compounds studied are shown in Table 2. Three of the better-known α₂-AR ligands (Idazoxan, Clonidine and RX821002) were used as references.

In the 2-aminoimidazoline series, the loss of the second cationic unit results in a remarkable loss of affinity towards the α₂-AR receptors. There is a drop of two logarithmic units in the case of 4b comparing to 1. The additional loss of the phenyl ring does not seem to be a very important structural change, since the pKᵢ values of 4b and 5b (see Table 2) can be considered within the same range and far from the affinity of the original lead compound. However, there is a trend change for compound 6b, as size reduction in the alkyl chain produces an increase in the affinity comparing to its counterparts 4b and 5b. Thus, its pKᵢ value, although not as good as that of the original lead, is higher than the Idazoxan and Clonidine pKᵢ values. As for 7b, the loss of the methyl group leads to a decrease in the affinity (Table 2), whose pK, value is the lowest within the 2-aminoimidazoline derivatives so far.

TABLE 2
Monocations prepared and their affinity for the α₂-ARs expressed as pKᵢ.
The structure of the reference compounds RX821002, Idazoxan and
Clonidine are also presented.

| Compound | pKᵢ |
|---|---|
| 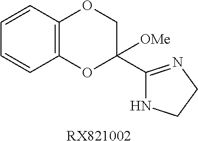 | 9.04 |
| 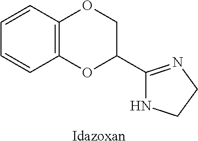 | 7.29 |
| 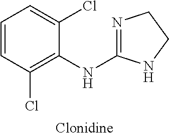 | 7.68 |
| 1 | 8.80 |
| 4b | 6.85 |
| 5b | 6.68 |
| 6b | 7.82 |
| 7b | 6.48 |
| 8b | 7.68 |
| 9b | 8.26 |
| 10b | 7.33 |
| 24 | 6.38 |
| 11b | 5.55 |
| 12b | 6.41 |
| 13b | 6.53 |
| 14b | 6.19 |
| 15b | 7.12 |
| 16b | 6.51 |
| 17b | 7.11 |

3,4-Disubstituted compounds 8b, 9b and 10b showed pKi >7 (see Table 2) and therefore within the range of Clonidine and/or Idazoxan, in fact, the affinity found for 9b is the second highest in this study.

We have previously observed⁹ that guanidine derivatives usually show lower affinities towards α₂-ARs than the 2-aminoimidazoline analogues. In this guanidine series, compounds 12b, 13b, 14b and 16b displayed similar pKᵢ values to the one showed by their dicationic analogue 24 (see Table 2), and 11b has the lowest affinity of the whole set of substrates described in this article. Remarkably, 15b and 17b, despite being guanidine derivatives, have an affinity within the range of Idazoxan. As expected, the pK, values found for all the guanidine derivatives were lower than the ones shown by their 2-aminoimidazoline analogues.

### Importance of the Methylene Substituent/Linker in Terms of the Affinity

A comparative analysis has been carried out to understand the difference in the affinity towards the receptors based on the steric and electronic properties of the chemical group in the linker. Thus, the α₂-AR pK, values of a number of these substrates previously reported by our group⁹ are displayed in Table 3 organized according to their structural similarities to those compounds presented in this work. Hence, for the PhXPhIm set, the affinity showed by 4b is slightly higher than the ones displayed by its analogues 25a, 26a and 27a (Table 3). This seems to indicate that for these structure-like compounds, the linker does not have a very important effect in the affinity for the receptor. As for the PhXPhGu-like compounds, the methylene linker is a drawback regarding the affinity, since the pKᵢ for 11b is lower than those of the compounds containing more electron-rich linkers. The most important difference in affinity (more than one logarithmic unit) is for compound 25b.

Regarding the CH₃XPhIm-like derivatives, again the presence of electron-rich groups seems to be an advantage (see 5b, 28a and 29a in Table 3), whereas for the CH₃XPhGu set there is not difference at all (see 12b, 28b and 29b in Table 3).

TABLE 3
Affinity values (pKᵢ) for the α₂-ARs of different sets of comparable structures with
different linkers

| Compd | Structure: PhXPhIm | pKᵢ | Compd | Structure: PhXPhGu | pKᵢ |
|---|---|---|---|---|---|
| 4b | 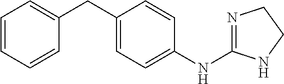 | 6.85 | 11b | 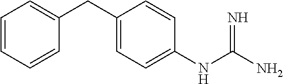 | 5.55 |
| 25a* | 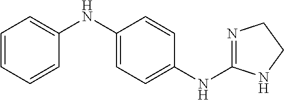 | 6.56 | 25b* | 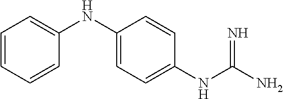 | 6.83 |
| 26a* | 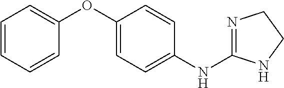 | 6.58 | 26b* | 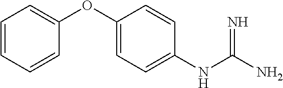 | 6.05 |
| 27a* | 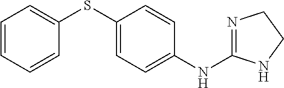 | 6.62 | 27b* | 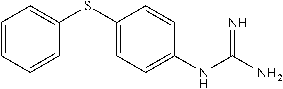 | 6.30 |
| Compd | Structure: CH₃XPhIm | pKᵢ | Compd | Structure: CH₃XPhGu | pKᵢ |
| 5b | 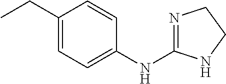 | 6.68 | 12b | 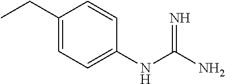 | 6.41 |
| 28a* | 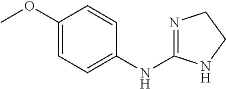 | 7.77 | 28b* | 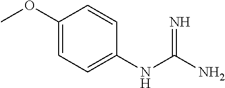 | 6.39 |
| 29a* | 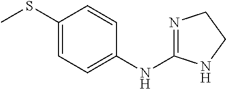 | 7.07 | 29b* | 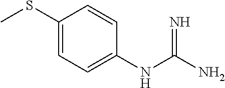 | 6.40 |
| Compd | Structure: XPhIm | pKᵢ | Compd | Structure: XPhGu | pKᵢ |
| 6b | 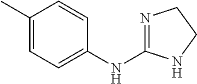 | 7.82 | 13b | 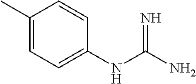 | 6.53 |
| 30a* | 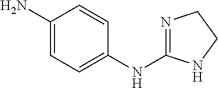 | 6.92 | 30b* | 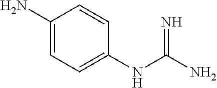 | 5.58 |
| Compd | Structure: 5-ringPhIm | pKᵢ | Compd | Structure: 5-ringPhGu | pKᵢ |
| 9b | 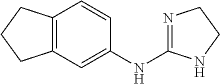 | 8.26 | 16b | 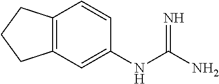 | 6.51 |
| 3* | 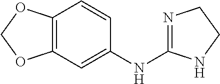 | 7.33 | 3b* | 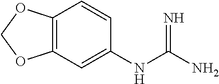 | 6.40 |
| Compd | Structure: 6-ringPhIm | pKᵢ | Compd | Structure: 6-ringPhGu | pKᵢ |
| 10b | 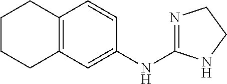 | 7.33 | 17b | 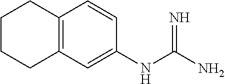 | 7.11 |
| 31a* | 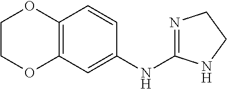 | 7.85 | 31b* | 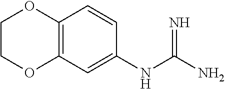 | 8.21 |

*The α₂-AR affinity of these compounds was reported in reference 9 by our group.

In the case of XPhIm and XPhGu analogues, the methyl derivatives showed pKᵢ values nearly one logarithmic unit higher than their amino counterparts (see Table 3), therefore, in this particular case, the presence of the alkyl group increases the affinity for the α₂-ARs.

Amongst the five-member ring containing derivatives, 5-ringPhIm, 9b displays a pKᵢ almost one logarithmic unit higher than the dioxo antagonist 3 (Table 3), thus, in this example the presence of the heteroatoms represents a burden affinity-wise. This difference is not so important in the guanidine set, 5-ringPhGu, since the pKᵢ values obtained for 16b and 3b are very similar.

In the case of the 6-ringPhIm and 6-ringPhGu-like structures, the presence of the oxygen atoms helps to increase the affinity towards the α₂-ARs, since 31a and 31b showed pKᵢ values higher than their methylene containing conterparts in 0.52 and 1.10 logarithmic units respectively (Table 3).

It can be concluded that the functionality in the linker does not seem to make a remarkable difference in the PhXPhIm, CH₃XPhGu or 5-ringPhGu-like structures, whereas the presence of an electron-rich group increases the affinity towards the α₂-ARs for the PhXPhGu, CH₃XPhIm, 6-ringPhIm and 6-ringPhGu sets. For the rest of structures, XPhIm, XPhGu, 5ringPhIm and the ImPhXPhIm twin substrates reported in our previous work,⁹ the alkyl group (methylene or methyl) has a positive effect in the α₂-AR affinity. No trend could be identified for the guanidine twin substrates GuPhXPhGu.⁹

### Affinity of the Dicationic Twin Compounds

Despite the fact that the monocationic compounds 6b, 8b, 9b, 10b, 15b and 17b showed interesting pKᵢ values, none of them increased the α₂-AR affinity of the original lead compound 1. Thus, we decided to prepare some twin substrates with different structural features. All these derivatives are shown in Table 4 alongside the pKᵢ values obtained in our study.

The conformationally constrained backbones of compounds 18b, 19b, 21b and 22b (see structures in Table 1), even though keep the lipophilic properties, reduce dramatically the rotation of the bonds around the bridge and, therefore, the cationic moieties spatial location will be restricted to limited areas. Thus, in a first approach, the comparison of their affinity to that of 1, will help to understand the range of distances between the cations required for a better interaction with the receptor. Additionally, if these conformationally restricted derivatives optimally interact with the receptor, this interaction will be energetically favoured since no energy would be spent in reaching the optimally oriented conformation. Conversely, 20b and 23b (see structures in Table 1) have more conformational freedom, and their study can also help to understand the dependence of the affinity on the intra-cations distance.

In the 2-aminoimidazoline series, none of these new substrates improved compound 1 affinity. However, 18b and 20b pKᵢ values (see Table 4) are within the range of Idazoxan and/or Clonidine and some interesting conclusions can be highlighted. For instance, the question arises whether the drop in the pKᵢ of 18b with respect to 1 is a consequence of the conformational constriction or to the fact that each one of the cations is now in meta and para positions with respect to each —CH₂— bridge. In the case of 19b, the drop in the affinity is even more remarkable (Table 4) indicating that to have both cations in meta respect to the linker is definitely a drawback. Nevertheless, more derivatives should be studied to fully evaluate the effect of the conformational restriction. As for compound 20b, the lengthening of the bridge resulted in more than one and a half logarithmic unit drop in the pKᵢ (Table 4) compared to 1, but still its affinity is close enough to that of Idazoxan.

TABLE 4
Twin molecules α₂-ARs affinity expressed as their pKᵢ

| Compound | Structure | pKᵢ |
|---|---|---|
| RX821002 | 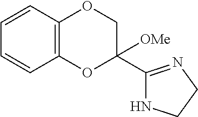 | 9.04 |
| Idazoxan | 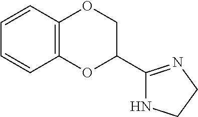 | 7.29 |
| Clonidine | 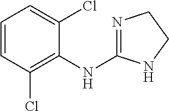 | 7.68 |
| 1 | 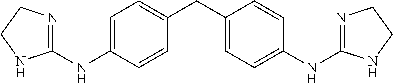 | 8.80 |
| 18b | 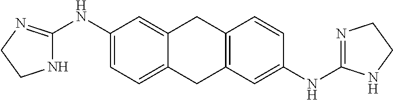 | 7.58 |
| 19b | 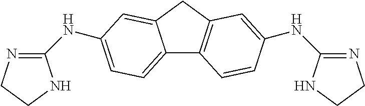 | 6.32 |
| 20b | 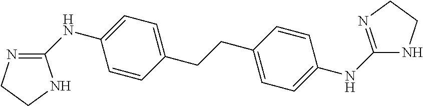 | 7.02 |
| 24 | 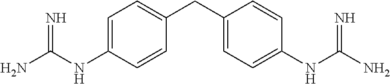 | 6.38 |
| 21b | 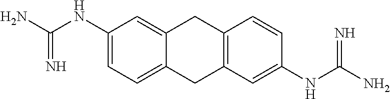 | 7.96 |
| 22b | 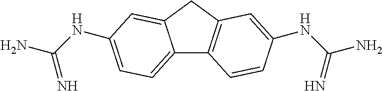 | 6.12 |
| 23b | 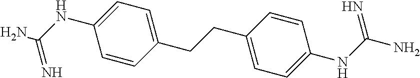 | 5.88 |

In the guanidine series, the most significant result is the good affinity obtained for 21 b (Table 4). Showing for the first time in this set of compounds better affinity than its 2-aminoimidazoline analogue. Actually, out of the compounds disclosed herein and previously reported in the literature by the present inventors this is the first example of a guanidine containing twin molecule showing a pKᵢ>7. Regarding 22b and 23b, both presented lower affinities than compound 24, however, unlike in the 2-aminoimidazoline series, the pKi obtained for the constricted analogue 22b is higher than the one found for 23b (Table 4). The relatively close pKi values found for the pairs of compounds 18b vs 21b and 19b vs 22b (see Table 4) could indicate that given a spatial environment, both, guanidinium and 2-aminoimidazolinium cations can have similar interactions with the receptors.¹⁹

### [³⁵S]GTPγs Binding Functional Assays

Those compounds which displayed an affinity at least within the range of Idazoxan and/or Clonidine (with a pKᵢ>7), were subject to [³⁵S]GTPγs binding experiments to determine their nature as agonists or antagonists.

As members of the G-protein coupled receptors (GPCRs) super-family, when the endogenous substrate binds to the α₂-ARs, they interact with a G-protein triggering a cascade of different biochemical events, which results in transmembrane signalling. This receptor activation alters the conformation of the G-proteins leading to the exchange of GDP by GTP on the α-subunit, promoting their dissociation into α-GTP and βγ subunits. A direct evaluation of this G-protein activity can be made by determining the guanine nucleotide exchange using radiolabelled GTP analogues.

The [³⁵S]GTPγs binding assay constitutes a functional measure of the interaction of the receptor and the G-protein and is a useful tool to distinguish between agonists (increasing the nucleotide binding), inverse agonists (decreasing the nucleotide binding), and neutral antagonists (not affecting the nucleotide binding) of GPCRs.²⁰ Experiments were performed in low-affinity receptor conditions for agonists (presence of guanine nucleotides and sodium in the medium), and therefore, typical potency values are two-three logarithmic units lower than affinity values obtained in radioligand receptor binding experiments.²⁰

Compounds 6b, 8b, 9b, 10b, and 15b stimulated binding of [³⁵S]GTPγs, showing a typical agonist dose-response plot. The potencies of all these substrates were in tillustrates their EC₅₀ values as well as their percentage efficacy relative to the well-known α₂-AR agonist UK14304. Conversely 17b, 18b, 20b and 21b did not stimulate binding of [³⁵S]GTPγs by their own and were subject to new [³⁵S]GTPγs binding experiments and tested against the α₂-AR agonist UK14304.

TABLE 5
Affinity for α₂-ARs (pKᵢ), EC₅₀ values and percentage efficacy relative to
UK14304 found for compounds showing a typical agonist dose-response plot.

| | | | EC₅₀ | |
|---|---|---|---|---|
| Compound | Structure | pKᵢ | microM) | Eₘₐₓ (%) |
| UK14304 | 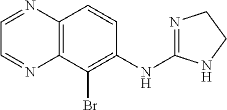 | 8.85 | 11.4 ± 0.3 | 100 |
| 6b | 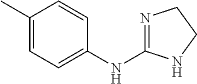 | 7.82 | 15.2 ± 0.2 | 97 |
| 8b | 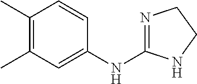 | 7.68 | 62.9 ± 0.7 | 89 |
| 9b | 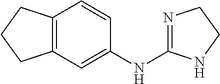 | 8.26 | 4.4 ± 0.3 | 98 |
| 10b | 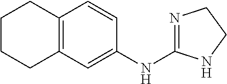 | 7.33 | 1.1 ± 0.1 | 100 |
| 15b | 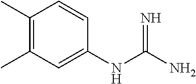 | 7.12 | 35.5 ± 0.8 | 84 |
| 4b | 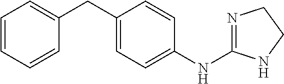 | 6.85 | 319 ± 17 | 111 |

In Table 6 can be found the effect induced in the UK14304 agonist stimulation of [³⁵S]GTPγs binding by the presence in the medium of a single concentration (10⁻⁵ M) of each of our compounds. Addition of 18b to the experiment did not induce a significant rightwards shift in the EC₅₀ value for the UK14304, whereas 21b resulted in a slight leftwards shift. These facts are in agreement with the lack of antagonistic properties towards the α₂-ARs for both substrates. On the contrary, 17b and 20b produced a remarkable rightwards shift in the UK14304 EC₅₀ value, result expected for antagonists. Hence, it is worth to highlight that, after these in vitro experiments in human brain PFC, two new antagonists with affinity similar to that of Idazoxan were obtained. Remarkably, and unexpectedly considering our previous results,⁹ 17b is the first guanidine containing compound that shows an antagonistic behaviour, whilst 20b is the first twin molecule of our “in-home library” displaying antagonistic features.

TABLE 6
EC₅₀ values obtained from the concentration-response curves for UK14304
stimulation of [³⁵S]GTPγS binding in the absence or presence of the different compounds
(10⁻⁵ M concentration).

| Experiment | Added compound structure | EC₅₀ (μM) |
|---|---|---|
| UK14304 | | 11.4 ± 0.3 |
| UK14304 + 17b | 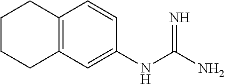 | 868.3 ± 112.8 |
| UK14304 + 18b | 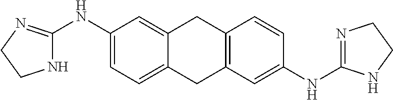 | 28.8 ± 4.2 |
| UK14304 + 20b | 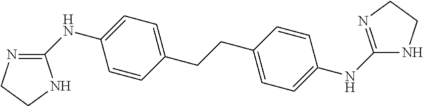 | 103.7 ± 10.8 |
| UK14304 + 21b | 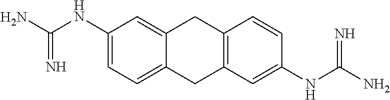 | 7.8 ± 0.3 |

### In Vivo Microdialysis Experiments

Considering the antagonistic properties and relatively good affinity over the α₂-ARs of compounds 17b and 20b, we tested their potential effect on noradrenergic transmission in vivo. Intracerebral microdialysis is a neurochemical technique that has been applied extensively in pharmacological studies aimed at investigating the effect of different drugs on brain neurotransmission. This technique allows one to collect a representative concentration of different neurotransmitters of the area where the probe is implanted while the animals are awake and freely moving.²¹

Most antidepressant drugs are able to increase NA extracellular concentrations in different brain areas as the PFC, an area implicated in depression disease. Considering that α₂-ARs exert a tonic inhibitory action on NA release from the noradrenergic terminals we assessed the ability of these two new compounds to increase NA extracellular concentration in this area. First, we tested the drugs when administered locally in order to confirm their antagonistic activity over α₂-ARs in vivo.

A second step was to study the effect of these compounds increasing NA extracellular concentrations when administered systemically.

Local administration of a CSF did not change NA basal values (F[8,30]=0.39; P=0.91, n=4, FIG. 2). However, reverse dialysis of 17b (1-100 μM) and 20b (1-100 μM) induced a concentration-related increase in extracellular NA levels (Emax=326±113%, (F[1,54]=8.05; P=0.0064, n=10; Emax=255±76%, F[1,46]=21.07; P<0.0001, n=7; respectively) (FIG. 2). The increases were very similar to those obtained from local administration (1-100 μM) of two well-known α₂-AR antagonists, RX821002 (Emax=287±49%; (F[1,391=66,78; P<0.0001, n=7) and Idazoxan (Emax=235±42%; (F[1,39]=32.07; P<0.0001, n=7) (FIG. 2).

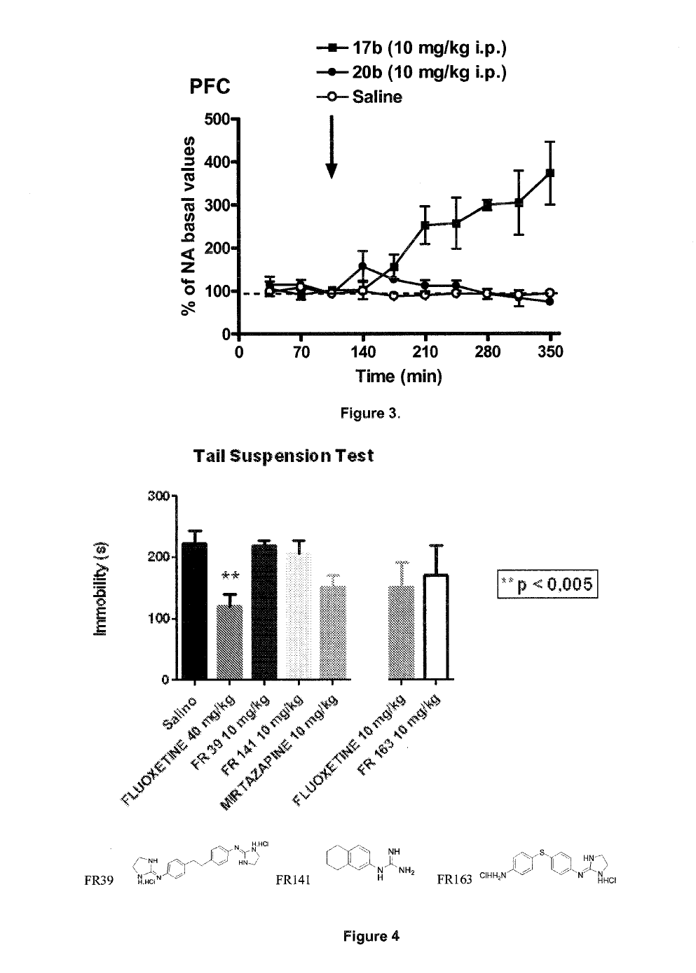

Systemic administration of 17b increased NA extracellular concentration by 373±73% and stayed high over the end of the experiment (F[1,33]=95.70; P<0.0001, n=6) (FIG. 4), whereas following administration of 20b a weak increase of NA basal values, reaching a maximal effect of 156±35%, was observed (FIG. 3). This increase was statistically significant when the group was compared with the respective control (F[1,30]=5.56; P=0.02, n=6).

Thus, 17b and 20b showed α₂-AR antagonist properties in vivo. Besides, both compounds were able to cross the blood brain barrier (BBB) as can be deduced by the increase evoked when they were systemically administered. However the stronger increase on NA basal values observed for 17b over 20b could indicate pharmacokinetic differences such as the ability to cross the BBB or differences in the catabolism of the substrates

### Experimental

### Pharmacology: Materials and Methods

Preparation of membranes. Neural membranes (P₂ fractions) were prepared from the PFC of human brains obtained at autopsy in the Instituto Vasco de Medicina Legal, Bilbao, Spain. Postmortem human brain samples of each subject (˜1 g) were homogenized using a Teflon-glass grinder (10 up-and-down strokes at 1500 rpm) in 30 volumes of homogenization buffer (1 mM MgCl₂, and 5 mM Tris-HCl, pH 7.4) supplemented with 0.25 M sucrose. The crude homogenate was centrifuged for 5 min at 1000×g (48° C.) and the supernatant was centrifuged again for 10 min at 40000×g (4° C.). The resultant pellet was washed twice in 20 volumes of homogenization buffer and recentrifuged in similar conditions. Aliquots of 1 mg protein were stored at −70° C. until assay. Protein content was measured according to the method Bradford using BSA as standard, and was similar in the different brain samples.

[³H]RX821002 binding assays. Specific [³H]RX821002 binding was measured in 0.55 ml-aliquots (50 mM Tris HCl, pH 7.5) of the neural membranes which were incubated with [³NRX821002 (1 nM) for 30 min at 25° C. in the absence or presence of the competing compounds (10⁻¹² M to 10⁻³ M, 10 concentrations). Incubations were terminated by diluting the samples with 5 ml of ice-cold Tris incubation buffer (4° C.). Membrane bound [³H]RX821002 was separated by vacuum filtration through Whatman GF/C glass fibre filters. Then, the filters were rinsed twice with 5 ml of incubation buffer and transferred to minivials containing 3 ml of OptiPhase “HiSafe” II cocktail and counted for radioactivity by liquid scintillation spectrometry. Specific binding was determined and plotted as a function of the compound concentration. Non-specific binding was determined in the presence of adrenaline (10⁻⁵ M).

Analysis of binding data. Analysis of competition experiments to obtain the inhibition constant (Kᵢ) were performed by nonlinear regression using the GraphPad Prism program. All experiments were analysed assuming a one-site model of radioligand binding. Kᵢ values were normalized to pKᵢ values.

[³⁵S]GTPγs binding assays. The incubation buffer for measuring [³⁵S]GTPγs binding to brain membranes contained, in a total volume of 500 μL, 1 mM EGTA, 3 mM MgCl₂, 100 mM NaCl, 50 mM GDP, 50 mM Tris-HCl at pH 7.4 and 0.5 nM [³⁵S]GTPγs. Proteins aliquots were thawed and re-suspended in the same buffer. The incubation was started by addition of the membrane suspension (40 μg of membrane proteins) to the previous mixture and was performed at 30° C. for 120 min with shaking. In order to evaluate the influence of the compounds on [³⁵S]GTPγs binding, 8 concentrations (10⁻¹⁰ to 10⁻³M) of the different compounds were added to the assay. Incubations were terminated by adding 3 mL of ice-cold re-suspension buffer followed by rapid filtration through Whatman GF/C filters pre-soaked in the same buffer. The filters were rinsed twice with 3 mL of ice-cold re-suspension buffer, transferred to vials containing 5 mL of OptiPhase HiSafe II cocktail (Wallac, UK) and the radioactivity trapped was determined by liquid scintillation spectrometry (Packard 2200CA). The [³⁵S]GTPγs bound was about 7-14% of the total [³⁵S]GTPγs added. Non-specific binding of the radioligand was defined as the remaining [³⁵S]GTPγs binding in the presence of 10 μM unlabelled GTPγs.

In Vivo Microdialysis assays. The experiments were carried out in male Sprague Dawley rats weighing between 250 g and 300 g. At the beginning of the experiments, animals were anaesthetized with chloral hydrate (400 mg/kg i.p.) and a microdialysis probe was implanted by stereotaxic surgery into prefrontal cortex (PFC) brain area. The coordinates selected for the PFC were as follows: AP (anterior to bregma) +2.8 mm, L (lateral from the mid-sagittal suture) +1 mm, DV (ventral from the dura surface) −5 mm.²² After 24 hours for animal recovery, perfusion fluid (artificial cerebrospinal fluid) is pumped through the probe at a flow rate of 1 μl/min. In the semipermeable membrane, that is the critical side of the probe and is placed on the selected area, molecules flow into and out the cannulae by diffusion. Therefore, microdialysis technique allows local administration of substrates dissolved in the perfusion fluids.

When drugs were locally administered, they were dissolved in a CSF and applied during 70 min via dialysis probe implanted in the PFC in increasing concentrations of 1, 10 and 100 μM. The compounds systemically administered were dissolved in saline and injected intraperitoneally.

Samples, collected with the microdialysis procedure (every 35 min), were analyzed by HPLC with electrochemical detection. NA concentrations were monitorizated by an amperometric detector (Hewlett-Packard model 1049A) at an oxidizing potential of +650 mV. The movil phase (12 mM citric acid, 1 mM EDTA, 0.7 mM octylsodio sulfate, pH 5 and 10% methanol) was filtered, degassed (Hewlett-Packard model 1100 degasser) and delivered at a flow rate of 0.2 ml/min by a Hewlett-Packard model 1100 pump. Stationary phase was a column of 150×2.1 mm (Thermo Electron Corporation, U.S.A.). Samples (injection volume 37 μl) were injected and NA analized in a run time of 10 min. Solution of standard noradrenaline was injected every working day to create a new calibration table.

The mean values of the first three samples before substrate administration were considered as 100% basal value. All measures of extracellular NA concentrations are expressed as percentage of the baseline value±s.e.mean. One way analysis of variance (ANOVA) for control group or two way ANOVA between control and each treated group was assessed for statistical analysis. After the experiments, rats were sacrificed with an overdose of chloral hydrate and the brains were dissected to check the correct implantation of the probe.

Drugs. [³H]RX821002 (specific activity 59 Ci/mmol) was obtained from Amersham International, UK. [³⁵S]GTPγS (1250 Ci/mmol) was purchased from DuPont NEN (Brussels, Belgium). Idazoxan HCl was synthesised by Dr. F. Geijo at S. A. Lasa Laboratories, Barcelona, Spain. Clonidine HCl, GDP, GTP, GTPγs, RX821002HCl, and UK14304 were purchased from Sigma (St. Louis, USA). All other chemicals were of the highest purity commercially available.

The tail suspension test as illustrated in FIG. 4 has become one of the most widely used models for assessing antidepressant-like activity in mice. The test is based on the fact that animals subjected to the short-term, inescapable stress of being suspended by their tail, will develop an immobile posture. Antidepressant medications encourage the animal to struggle and promote the occurrence of escape-related behaviour. As illustrated in FIG. 4, administration of compounds 17b and 20b resulted in slightly shorter immobility period than the saline control.

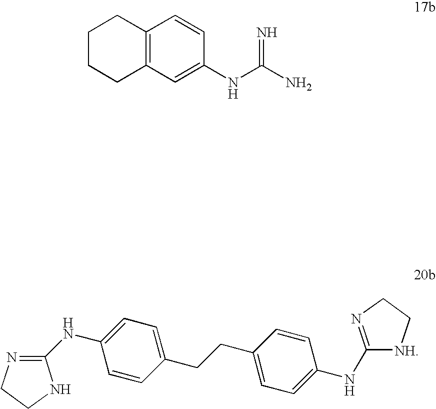

### Chemistry

All the commercial chemicals were obtained from Sigma-Aldrich or Fluka and were used without further purification. Deuterated solvents for NMR use were purchased from Apollo. Dry solvents were prepared using standard procedures, according to Vogel, with distillation prior to use. Chromatographic columns were run using Silica gel 60 (230-400 mesh ASTM) or Aluminium Oxide (activated, Neutral Brockman I STD grade 150 mesh). Solvents for synthesis purposes were used at GPR grade. Analytical TLC was performed using Merck Kieselgel 60 F₂₅₄ silica gel plates or Polygram Alox N/U V₂₅₄ aluminium oxide plates. Visualisation was by UV light (254 nm). NMR spectra were recorded in a Bruker DPX-400 Avance spectrometer, operating at 400.13 MHz and 600.1 MHz for ¹H-NMR and 100.6 MHz and 150.9 MHz for ¹³C-NMR. Shifts are referenced to the internal solvent signals. NMR data were processed using Bruker Win-NMR 5.0 software. Electrospray mass spectra were recorded on a Mass Lynx NT V 3.4 on a Waters 600 controller connected to a 996 photodiode array detector with methanol, water or ethanol as carrier solvents. Melting points were determined using an Electrothermal IA9000 digital melting point apparatus and are uncorrected. Infrared spectra were recorded on a Mattson Genesis II FTIR spectrometer equipped with a Gateway 2000 4DX2-66 workstation and on a Perkin Elmer Spectrum One FT-IR Spectrometer equipped with Universal ATR sampling accessory. Sample analysis was carried out in nujol using NaCl plates. Elemental analysis was carried out at the Microanalysis Laboratory, School of Chemistry and Chemical Biology, University College Dublin.

### General Procedure for the Synthesis of Boc-Protected Guanidine Derivatives: Method A.

Each of the corresponding amines (or diamines) was treated either in DCM or DMF at 0° C. with 1.1 equivalents (or 2.2 for the diamines) of mercury (II) chloride, 1.0 equivalents (or 2.0 for the diamines) of NN-di(tert-butoxycarbonyl)thiourea and 3.1 equivalents (or 5.0 for the diamines) of TEA. The resulting mixture was stirred at 0° C. for 1 hour and for the appropriate duration at room temperature. Then, the reaction mixture was diluted with EtOAc and filtered through a pad of Celite to get rid of the mercury sulfide formed. The filter cake was rinsed with EtOAc. The organic phase was extracted with water (2×30 mL), washed with brine (1×30 mL), dried over anhydrous Na₂SO₄ and concentrated under vacuum to give a residue that was purified by silica gel column chromatography, eluting with the appropriate hexane:EtOAc mixture.

### General Procedure for the Synthesis of the Boc-Protected 2-Iminoimidazolidine Derivatives: Method B.

Each of the corresponding amines (or diamines) was treated either in DCM or DMF at 0° C. with 1.1 equivalents (or 2.2 for the diamines) of mercury (II) chloride, 1.0 equivalents (or 2.0 for the diamines) of N,N′-di(tert-butoxycarbonyl)imidazolidine-2-thione and 3.1 equivalents (or 5.0 for the diamines) of TEA. The resulting mixture was stirred at 0° C. for 1 hour and for the appropriate duration at room temperature. Then, the reaction mixture was diluted with EtOAc and filtered through a pad of Celite to get rid of the mercury sulfide formed. The filter cake was rinsed with EtOAc. The organic phase was extracted with water (2×30 mL), washed with brine (1×30 mL), dried over anhydrous Na₂SO₄ and concentrated under vacuum to give a residue that was purified by neutral alumina column flash chromatography, eluting with the appropriate hexane:EtOAc mixture. The residue obtained after the column was recrystallised from the appropriate solvent when required.

### General Procedure for the Synthesis of the Hydrochloride Salts: Method C.

Each of the corresponding Boc-protected precursors (0.5 mmol) was treated with 15 mL of a 50% solution of trifluoroacetic acid in DCM for 3 h. After that time, the solvent was eliminated under vacuum to generate the trifluoroacetate salt. This salt was dissolved in 20 mL of water and treated for 24 h with IRA400 Amberlyte resin in its Cl⁻ form. Then, the resin was removed by filtration and the aqueous solution washed with DCM (2×10 mL). Evaporation of the water afforded the pure hydrochloride salt. Absence of the trifluoroacetate salt was checked by ¹⁹F NMR.

### 1-[1,3-di(tert-butoxycarbonyI)-2-imidazolidinylimino]-4-benzyl benzene (4a): Method B

896 mg (3.3 mmol) of HgCl₂ were added over a solution of 550 mg (3.0 mmol) of 4-benzyl-aniline, 907 mg (3.0 mmol) of N,N′-di(tert-butoxycarbonyl)imidazolidine-2-thione and 1.3 mL (9.3 mmol) of TEA in DCM (5 mL) at 0° C. The resulting mixture was stirred at 0° C. for 1 h and 22 h more at room temperature. Usual work up followed by neutral alumina column flash chromatography, eluting with hexane:EtOAc (2:1) gave a residue which was recrystallised from hexane to afford 4a as a white solid (1052 mg, 78% yield); mp 124-126° C. Hydrochloride salt of 1-(2-imidazolidinyliminio)-4-benzylbenzene (4b): Method C.

Yellowish oil (96%); ¹H NMR (D₂O) δ 3.55 (s, 4H), 3.57 (s, 2H), 6.87-7.04 (m, 9H); ¹³C NMR (D₂O) δ 40.2, 42.1, 122.9, 125.5, 128.0, 128.2, 129.4, 132.6, 139.5, 140.6, 157.4; MS (ESI⁺) m/z 252.0944 [M+H]⁺. Anal. (C₁₆H₁₈ClN₃.1.4H₂O) C, H, N.

### 1-[1,3-di(tert-butoxycarbonyl)-2-imidazolidinylimino]-4-ethylbenzene (5a): Method B

896 mg (3.3 mmol) of HgCl₂ were added over a solution of 364 mg (3.0 mmol) of 4-ethyl-phenylamine, 907 mg (3.0 mmol) of N,N′-di(tert-butoxycarbonyl)imidazolidine-2-thione and 1.3 mL (9.3 mmol) of TEA in DCM (5 mL) at 0° C. The resulting mixture was stirred at 0° C. for 1 h and 25 h more at room temperature. Usual work up followed by neutral alumina column flash chromatography, eluting with hexane:EtOAc (2:1) gave a residue which was recrystallised from hexane to afford 5a as a white solid (942 mg, 80% yield); mp 89-91° C.

### 1-[1,3-di(tert-butoxycarbonyl)-2-imidazolidinylimino]-4-methylbenzene (6a): Method B

896 mg (3.3 mmol) of HgCl₂ were added over a solution of 322 mg (3.0 mmol) of p-tolylamine, 907 mg (3.0 mmol) of N,N′-di(tert-butoxycarbonyl)imidazolidine-2-thione and 1.3 mL (9.3 mmol) of TEA in DCM (5 mL) at 0° C. The resulting mixture was stirred at 0° C. for 1 h and 20 h more at room temperature. Usual work up followed by neutral alumina column flash chromatography, eluting with hexane:EtOAc (4:3) gave a residue which was recrystallised from hexane to afford 6a as a white solid (781 mg, 69% yield); mp 105-107° C.

### [1,3-di(tert-butoxycarbonyI)-2-imidazolidinylimino]benzene (7a): Method B

896 mg (3.3 mmol) of HgCl₂ were added over a solution of 280 mg (3.0 mmol) of aniline, 907 mg (3.0 mmol) of N,N′-di(tert-butoxycarbonyl)imidazolidine-2-thione and 1.3 mL (9.3 mmol) of TEA in DCM (5 mL) at 0° C. The resulting mixture was stirred at 0° C. for 1 h and 19 h more at room temperature. Usual work up followed by neutral alumina column flash chromatography, eluting with hexane:EtOAc (2:1) gave 7a as a white solid (890 mg, 82% yield); mp 142-144° C.

### 1-[1,3-di(tert-butoxycarbonyl)-2-imidazolidinylimino]-3,4-dimethylbenzene (8a): Method B

896 mg (3.3mmol) of HgCl₂ were added over a solution of 364mg (3.0 mmol) of 3,4-dimethyl-phenylamine, 907 mg (3.0 mmol) of NN-di(tert-butoxycarbonyl)imidazolidine-2-thione and 1.3 mL (9.3 mmol) of TEA in DCM (5 mL) at 0° C. The resulting mixture was stirred at 0° C. for 1 h and 20 h more at room temperature. Usual work up followed by neutral alumina column flash chromatography, eluting with hexane:EtOAc (5:2) gave a residue which was recrystallised from hexane to afford 8a as a white solid (724 mg, 62% yield); mp 102-104° C.

### Hydrochloride salt of 1-(2-imidazolidinylimino)-3,4-dimethylbenzene (8b): Method C

White solid (96%); mp 83-85° C.; ¹H NMR (D₂O) δ 2.08 (s, 6H), 3.58 (s, 4H), 6.80 (d, 1H, J=8.0 Hz), 6.83 (s, 1H), 7.06 (d, 1H, J=8.0 Hz); ¹³C NMR (D₂O) δ 17.9, 18.4, 42.1, 120.1, 123.7, 130.0, 131.9, 135.4, 138.1, 157.5; MS (ESI⁺) m/z 190.1163 [M+H]⁺. Anal. (C₁₁H₁₆ClN₃.0.2H₂O) C, H, N.

### 5-[1,3-di(tert-butoxycarbonyl)-2-imidazolidinylimino]indan (9a): Method B

896 mg (3.3 mmol) of HgCl₂ were added over a solution of 400 mg (3.0 mmol) of 5-aminoindan, 907 mg (3.0 mmol) of N,N′-di(tert-butoxycarbonyl)imidazolidine-2-thione and 1.3 mL (9.3 mmol) of TEA in DCM (5 mL) at 0° C. The resulting mixture was stirred at 0° C. for 1 h and 18 h more at room temperature. Usual work up followed by neutral alumina column flash chromatography, eluting with hexane:EtOAc (5:2) gave a residue which was recrystallised from hexane to afford 9a as a white solid (780 mg, 65% yield); mp 123-124° C.

### 2-(5,6,7,8-Tetrahydro-naphthalen-2-ylimino)-imidazolidine-1,3-dicarboxylic acid di-tert-butyl ester (10a): Method B

896 mg (3.3 mmol) of HgCl₂ were added over a solution of 442 mg (3.0 mmol) of 5,6,7,8-tetrahydro-naphtalen-2-ylamine, 907 mg (3.0 mmol) of N,A′-di(tert-butoxycarbonyl)imidazolidine-2-thione and 1.3 mL (9.3 mmol) of TEA in DCM (5 mL) at 0° C. The resulting mixture was stirred at 0° C. for 1 h and 20 h more at room temperature. Usual work up followed by neutral alumina column flash chromatography, eluting with (3:1) gave 10a as a white solid (988 mg, 79% yield); mp 140-142° C.

### Hydrochloride salt of imidazolidin-2-ylidene-(5,6,7,8-tetrahydro-naphthalen-2-yl)-amine (10b): Method C

White solid (94%); mp 87-89° C.; ¹H NMR (D₂O) δ 1.65-1.74 (m, 4H), 2.63-2.75 (m, 4H), 3.70 (s, 4H), 6.88-6.99 (m, 2H), 7.13 (d, 1H, J=8.0 Hz); ¹³C NMR (D₂O) δ 21.8, 22.0, 27.9, 28.3, 42.2, 120.7, 123.9, 129.8, 131.7, 136.4, 138.6, 158.1; MS (ESI⁺) m/z 216.1380 [M+H]⁺. Anal. (C₁₁H₁₆ClN₃.1.3H₂O) C, H, N.

### 1-[2,3-di(tert-butoxycarbonyl)guanidino]-4-benzylbenzene (11a): Method A

896 mg (3.3 mmol) of HgCl₂ were added over a solution of 550 mg (3.0 mmol) of 4-benzyl-aniline, 830 mg (3.0 mmol) of N,N′-di(tert-butoxycarbonyl)thiourea and 1.3 mL (9.3 mmol) of TEA in DCM (5 mL) at 0° C. The resulting mixture was stirred at 0° C. for 1 h and 19 h more at room temperature. Usual work up followed by silica gel column chromatography, eluting with hexane:EtOAc (5:2) gave 11a as a white solid (1.035 mg, 81% yield); mp 116-118° C.

### 1-[2,3-di(tert-butoxycarbonyl)guanidino]-4-ethylbenzene (12a): Method A

896 mg (3.3 mmol) of HgCl₂ were added over a solution of 364 mg (3.0 mmol) of 4-ethyl-phenylamine, 830 mg (3.0 mmol) of N,N′-di(tert-butoxycarbonyl)thiourea and 1.3 mL (9.3 mmol) of TEA in DCM (5 mL) at 0° C. The resulting mixture was stirred at 0° C. for 1 h and 18 h more at room temperature. Usual work up followed by silica gel column chromatography, eluting with hexane:EtOAc (4:1) gave 12a as an orange solid (843 mg, 77% yield); mp 99-101° C.

### 1-[2,3-di(tert-butoxycarbonyl)guanidino]-4-methylbenzene (13a): Method A

896 mg (3.3 mmol) of HgCl₂ were added over a solution of 322 mg (3.0 mmol) of p-tolyl-amine, 830 mg (3.0 mmol) of N,N′-di(tert-butoxycarbonyl)thiourea and 1.3 mL (9.3 mmol) of TEA in DCM (5 mL) at 0° C. The resulting mixture was stirred at 0° C. for 1 h and 23 h more at room temperature. Usual work up followed by silica gel column chromatography, eluting with hexane:EtOAc (3:1) gave 13a as a white solid (850 mg, 81% yield); mp 120-122° C.

### 1-[2,3-di(tert-butoxycarbonyl)guanidino]-3,4-dimethylbenzene (15a): Method A

896 mg (3.3 mmol) of HgCl₂ were added over a solution of 364 mg (3.0 mmol) of 3,4-dimethyl-phenylamine, 830 mg (3.0 mmol) of N,N′-di(tert-butoxycarbonyl)thiourea and 1.3 mL (9.3 mmol) of TEA in DCM (5 mL) at 0° C. The resulting mixture was stirred at 0° C. for 1 h and 16 h more at room temperature. Usual work up followed by silica gel column chromatography, eluting with hexane:EtOAc (5:2) gave 15a as a white solid (937 mg, 86% yield); mp 124-126° C.

### 5-[2,3-di(tert-butoxycarbonyl)guanidino]indan (16a): Method A

896 mg (3.3 mmol) of HgCl₂ were added over a solution of 400 mg (3.0 mmol) of 5-amino-indan, 830 mg (3.0 mmol) of N,N′-di(tert-butoxycarbonyl)thiourea and 1.3 mL (9.3 mmol) of TEA in DCM (5 mL) at 0° C. The resulting mixture was stirred at 0° C. for 1 h and 16 h more at room temperature. Usual work up followed by silica gel column chromatography, eluting with hexane:EtOAc (3:1) gave 16a as a yellowish solid (916 mg, 81% yield); mp 103-105° C.

### 6-[2,3-di(tert-butoxycarbonyl)guanidino]-1,2,3,4-tetrahydronaphtalene (17a): Method A

896 mg (3.3 mmol) of HgCl₂ were added over a solution of 442 mg (3.0 mmol) of 5,6,7,8-tetrahydro-naphtalen-2-ylamine, 830 mg (3.0 mmol) of N,N′-di(tert-butoxycarbonyl)thiourea and 1.3 mL (9.3 mmol) of TEA in DCM (5 mL) at 0° C. The resulting mixture was stirred at 0° C. for 1 h and 16 h more at room temperature. Usual work up followed by silica gel column chromatography, eluting with hexane:EtOAc (3:1) gave 17a as a white solid (915 mg, 78% yield); mp 122-124° C.

### Hydrochloride salt of N-(5,6,7,8-tetrahydro-naphthalen-2-yl)-guanidine (17b): Method C

White solid (95%); mp 39-41° C.; ¹H NMR (D₂O) δ 1.58-1.72 (m, 4H), 2.59-2.72 (m, 4H), 6.81-6.93 (m, 2H), 7.07 (d, 1H, J=8.0 Hz); ¹³C NMR (D₂O) δ 21.9, 22.0, 28.0, 28.2, 122.1, 125.4, 129.9, 130.6, 136.7, 138.6, 155.6; MS (ESI⁺) m/z 190.1248 [M+H]⁺. Anal. (C₁₁H₁₆ClN₃.0.8H₂O) C, H, N.

### 2,6-Bis[1,3-di(tert-butoxycarbonyl)-2-imidazolidinylimino]-9,10-dihydroanthracene (18a): Method B

896 mg (3.3 mmol) of HgCl₂ were added over a solution of 315 mg (1.5 mmol) of 2,6-diamino-9,10-dihydroanthracene, 907 mg (3.0 mmol) of N,N′-di(tert-butoxycarbonyl)-imidazolidine-2-thione and 1.3 mL (9.3 mmol) of TEA in DMF (5 mL) at 0° C. The resulting mixture was stirred at 0° C. for 1 h and 48 h more at room temperature. Usual work up followed by neutral alumina column flash chromatography, eluting with hexane:EtOAc (1:1) gave 18a as a yellow solid (788 mg, 70% yield); mp 208-210° C.

### Dihydrochloride salt of 2,6-di(2-imidazolidinylimino)-9,10-dihydroanthracene (18b): Method C

Brown solid (94%); mp decomposes over 220° C.; ¹H NMR (D₂O) δ 3.66 (s, 8H), 3.73 (s, 4H), 6.92-7.06 (m, 4H), 7.24 (d, 2H, J=8.0 Hz); ¹³C NMR (D₂O) δ 34.1, 42.2, 120.9, 121.8, 128.0, 132.4, 134.6, 137.5, 157.8; MS (ESI⁺) m/z 347.1572 [M+H]⁺. Anal. (C₂₀H₂₄Cl₂N₆.2.0H₂O) C, H, N.

### 2,7-Bis[1,3-di(tert-butoxycarbonyl)-2-imidazolidinylimino]-9H-fluorene (19a): Method B

896 mg (3.3 mmol) of HgCl₂ were added over a solution of 294 mg (1.5 mmol) of 2,7-diaminofluorene, 907 mg (3.0 mmol) of NN-di(tert-butoxycarbonyl)imidazolidine-2-thione and 1.3 mL (9.3 mmol) of TEA in DCM (5 mL) at 0° C. The resulting mixture was stirred at 0° C. for 1 h and 24 h more at room temperature. Usual work up followed by neutral alumina column flash chromatography, eluting with hexane:EtOAc (1:1) gave a residue which was recrystallised from Et₂O to afford 19a as a white solid (779 mg, 71% yield); mp 190-192° C.

### Dihydrochloride salt of 2,7-di(2-imidazolidinylimino)-9H-fluorene (19b): Method C.

Light brown solid (95%); mp decomposes over 240° C.; ¹H NMR (D₂O) δ 3.64 (s, 8H), 3.68 (s, 2H), 7.06 (d, 2H, J=8.0 Hz), 7.22 (s, 2H), 7.64 (d, 2H, J=8.0 Hz); ¹³C NMR (D₂O) δ 35.9, 42.1, 119.1, 120.3, 121.2, 133.2, 138.3, 144.5, 157.3; MS (ESI⁺) m/z 333.1827 [M+H]⁺. Anal. (C₁₉H₂₂Cl₂N₆.1.8H₂O) C, H, N.

### 4,4′-Bis[1,3-di(tert-butoxycarbonyl)-2-imidazolidinylimino]-1,2-diphenylethane (20a): Method B

896 mg (3.3 mmol) of HgCl₂ were added over a solution of 318 mg (1.5 mmol) of 4,4′-diaminobibenzyl, 907 mg (3.0 mmol) of NN-di(tert-butoxycarbonyl)imidazolidine-2-thione and 1.3 mL (9.3 mmol) of TEA in DCM (5 mL) at 0° C. The resulting mixture was stirred at 0° C. for 1 h and 28 h more at room temp. Usual work up followed by neutral alumina column flash chromatography, eluting with hexane:EtOAc (2:3) gave a residue which was precipitated with cold hexane to give 20a as a white solid (800 mg, 71% yield); mp 196-198° C.

### Dihydrochloride salt of 4,4′-di(2-imidazolidinylimino)-1,2-diphenylethane (20b): Method C

Yellowish solid (96%); mp decomposes over 210° C.; ¹H NMR (D₂O) δ 2.91 (s, 4H), 3.70 (s, 8H), 7.13 (d, 4H, J=8.0 Hz), 7.25 (d, 4H, J=8.0 Hz); ¹³C NMR (D₂O) δ 35.5, 42.2, 123.6, 129.4, 132.3, 140.4, 158.1; MS (ESI⁺) m/z 349.1840 [M+H]⁺. Anal. (C₂₀H₂₆Cl₂N₆.1.3H₂O) C, H, N.

### 2,6-Bis[2,3-di(tert-butoxycarbonyl)guanidino]-9,10-dihydroanthracene (21a): Method A

706 mg (2.6 mmol) of HgCl₂ were added over a solution of 250 mg (1.2 mmol) of 2,6-diamino-9,10-dihydroanthracene, 663 mg (2.4 mmol) of N,N′-di(tert-butoxycarbonyl)thiourea and 1 mL (7.1 mmol) of TEA in DMF (5 mL) at 0° C. The resulting mixture was stirred at 0° C. for 1 h and 25 h more at room temperature. Usual work up followed by silica gel column chromatography, eluting with hexane:EtOAc (5:2) gave 21a as a white solid (508 mg, 61% yield); mp decomposes over 235° C.

### Dihydrochloride salt of 2,6-diguanidino-9,10-dihydroanthracene (21 b): Method C

Brown solid (95%); mp decomposes over 215° C.; ¹H NMR (D₂O) δ 3.81 (s, 4H), 7.06 (d, 2H, J=8.0 Hz), 7.10 (s, 2H), 7.31 (d, 2H, J=8.0 Hz); ¹³C NMR (D₂O) δ 34.2, 122.9, 123.8, 128.1, 131.5, 135.5, 137.7, 155.7; MS (ESI⁺) m/z 295.1659 [M+H]⁺. Anal. (C₁₆H₂₀Cl₂N₆.2.3H₂O) C, H, N.

### 4,4′-Bis[2,3-di(tert-butoxycarbonyl)guanidino]-1,2-diphenylethane (23a): Method A

896 mg (3.3 mmol) of HgCl₂ were added over a solution of 318 mg (1.5 mmol) of 4,4′-diamino-bibenzyl, 829 mg (3.0 mmol) of N,N′-di(tert-butoxycarbonyl)thiourea and 1.3 mL (9.3 mmol) of TEA in DCM (5 mL) at 0° C. The resulting mixture was stirred at 0° C. for 1 h and 25 h more at room temperature. Usual work up followed by silica gel column chromatography, eluting with hexane:EtOAc (2:1) gave 23a as a white solid (650 mg, 62% yield); mp>300° C.

Supporting Information Available. IR, ¹H NMR, ¹³C NMR and MS data for the compounds already described in the literature (5b-7b, 9b, 11b-13b, 14a, 14b-16b, 22a, 22b and 23b) and all new Boc-protected derivatives prepared (4a-13a, 15a-21a and 23a). A table containing the combustion analysis data for the new final compounds (4b, 8b, 10b and 17b-21b) is also presented.

### Compounds Previously Prepared:

Hydrochloride salt of 1-(2-imidazolidinylimino)-4-ethylbenzene or hydrochloride salt of (4-ethyl-phenyl)-imidazolidin-2-ylidene-amine (5b): Method C.

Brownish oil (95%); ¹H NMR (D₂O) δ 1.11 (t, 3H, J=7.5 Hz), 2.54 (q, 2H, J=7.5 Hz), 3.66 (s, 4H), 7.08 (d, 2H, J=7.0 Hz), 7.22 (d, 2H, J=7.0 Hz); ¹³C NMR (D₂O) δ 14.5, 27.3, 42.2, 123.1, 128.7, 132.0, 143.2, 157.6; MS (ESI⁺) m/z 190.1334 [M+H]⁺.

### Hydrochloride salt of 1-(2-imidazolidinylimino)-4-methylbenzene or hydrochloride salt of imidazolidin-2-ylidene-p-tolyl-amine (6b): Method C

Greenish solid (94%); mp 153-155° C.; ¹H NMR (D₂O) δ 2.30 (s, 3H), 3.66 (s, 4H), 7.09 (d, 2H, J=8.0 Hz), 7.25 (d, 2H, J=8.0 Hz); ¹³C NMR (D₂O) δ 19.7, 42.2, 123.3, 129.8, 131.7, 137.2, 157.8; MS (ESI⁺) m/z 176.1192 [M+H]⁺.

### Hydrochloride salt of (2-imidazolidinylimino)benzene or hydrochloride salt of imidazolidin-2-ylidene-phenyl-amine (7b): Method C

White solid (97%); mp 211-213° C.; ¹H NMR (D₂O) δ 3.74 (s, 4H), 7.27-7.32 (m, 2H), 7.35-7.41 (m, 1H), 7.44-7.50 (m, 2H); ¹³C NMR (D₂O) δ 42.2, 123.9, 127.0, 129.3, 134.6, 158.4; MS (ESI⁺) m/z 162.1021 [M+H]⁺.

### Hydrochloride salt of 5-(2-imidazolidinylimino)indan or hydrochloride salt of imidazolidin-2-ylidene-indan-5-yl-amine (9b): Method C

Light brown solid (95%); mp 183-185° C.; ¹H NMR (D₂O) δ 2.00-2.13 (m, 2H), 2.86-2.94 (m, 4H), 3.73 (s, 4H), 7.04 (d, 1H, J=8.0 Hz), 7.17 (s, 1H), 7.32 (d, 1H, J=8.0 Hz); ¹³C NMR (D₂O) δ 24.8, 31.4, 31.8, 42.2, 119.6, 121.4, 124.8, 132.4, 143.4, 146.0, 158.1; MS (ESI⁺) m/z 202.1346 [M+H]⁺.

### Hydrochloride salt of 1-guanidino-4-benzylbenzene or hydrochloride salt of N-(4-benzyl-phenyl)-guanidine (11 b): Method C

Yellowish oil (97%); ¹H NMR (D₂O) δ 3.64 (s, 2H), 6.89-7.08 (m, 9H); ¹³C NMR (D₂O) δ 40.2, 125.0, 125.6, 128.1, 128.2, 129.5, 131.5, 140.3, 140.6, 155.4; MS (ESI⁺) m/z 226.0974 [M+H]⁺.

### Hydrochloride salt of 1-guanidino-4-ethylbenzene or hydrochloride salt of N-(4-ethyl-phenyl)-guanidine (12b): Method C

Light brown solid (94%); mp 97-99° C.; ¹H NMR (D₂O) δ 1.11 (t, 3H, J=7.5 Hz), 2.55 (q, 2H, J=7.5 Hz), 7.09 (d, 2H, J=8.0 Hz), 7.23 (d, 2H, J=8.0 Hz); ¹³C NMR(D₂O) δ 14.5, 27.4, 125.2, 128.8, 130.9, 144.1, 155.7; MS (ESI⁺) m/z 164.0943 [M+H]⁺.

### Hydrochloride salt of 1-guanidino-4-methylbenzene or hydrochloride salt of N-p-tolyl-guanidine (13b): Method C

Yellow solid (98%); mp 129-131° C.; ¹H NMR (D₂O) 6 2.21 (s, 3H), 6.98 (d, 2H, J=8.0 Hz), 7.14 (d, 2H, J=8.0 Hz); ¹³C NMR (D₂O) δ 19.8, 124.9, 130.0, 130.6, 137.7, 155.5; MS (ESI⁺) m/z 150.0744 [M+H]⁺.

### 1-[2,3-di(tert-butoxycarbonyl)guanidino]benzene (14a): Method A

896 mg (3.3 mmol) of HgCl₂ were added over a solution of 280 mg (3.0 mmol) of aniline, 830 mg (3.0 mmol) of N,N′-di(tert-butoxycarbonyl)thiourea and 1.3 mL (9.3 mmol) of TEA in DCM (5 mL) at 0° C. The resulting mixture was stirred at 0° C. for 1 h and 16 h more at room temp. Usual work up followed by silica gel column chromatography, eluting with hexane:EtOAc (5:1) gave 14a as a white solid (736 mg, 73% yield); mp 119-121° C.; IR (nujol) v 3262, 3193, 1727, 1636 cm⁻¹; ¹H NMR (CDCl₃) δ 1.51 (s, 9H), 1.54 (s, 9H), 7.03-7.16 (m, 1H), 7.28-7.36 (m, 2H), 7.61 (d, 2H, J=8.0 Hz), 10.34 (br, 1H), 11.66 (br, 1H); ¹³C NMR (CDCl₃) δ 27.9, 28.0, 79.4, 83.5, 122.0, 124.6, 128.7, 136.6, 153.2, 153.4, 163.4.

### Hydrochloride salt of N-phenyl-guanidine (14b): Method C

Brown oil (96%); ¹H NMR (D₂O) δ 7.27 (d, 2H, J=7.5 Hz), 7.41 (‘t’, 1H, J=7.5 Hz), 7.50 (‘t’, 2H, J=7.5 Hz); ¹³C NMR (D₂O) δ 125.2, 127.5, 129.6, 133.5, 155.6; MS (ESI⁺) m/z 136.0872 [M+H]⁺.

### Hydrochloride salt of 1-guanidino-3,4-dimethylbenzene or hydrochloride salt of N-(3,4-dimethyl-phenyl)-guanidine (15b): Method C

Yellow solid (97%); mp 115-117° C.; ¹H NMR (D₂O) δ 2.16 (s, 3H), 2.17 (s, 3H), 6.91 (d, 1H, J=8.0 Hz), 6.94 (s, 1H), 7.16 (d, 1H, J=8.0 Hz); ¹³C NMR (D₂O) δ 18.0, 18.4, 122.22, 125.8, 130.2, 130.9, 136.4, 138.3, 155.6; MS (ESI⁺) m/z 164.0934 [M+H]⁺.

### Hydrochloride salt of 5-guanidinoindan or hydrochloride salt of N-indan-5-yl-guanidine (16b): Method C

Brown solid (96%); mp 51-53° C.; ¹H NMR (D₂O) δ 1.95-2.07 (m, 2H), 2.79-2.92 (m, 4H), 6.98 (d, 1H, J=8.0 Hz), 7.09 (s, 1H), 7.28 (d, 1H, J=8.0 Hz); ¹³C NMR (D₂O) δ 24.8, 31.5, 31.8, 121.4, 123.2, 124.9, 131.3, 144.2, 146.1, 155.9; MS (ESI⁺) m/z 176.1185 [M+H]⁺.

### 2,7-Bis[N′,N″-di(tert-butoxycarbonyl)guanidino]-9H-fluorene (22a): Method A

896 mg (3.3 mmol) of HgCl₂ were added over a solution of 295 mg (1.5 mmol) of 9H-fluorene-2,7-diamine, 829 mg (3.0 mmol) of N,N′-di(tert-butoxycarbonyl)thiourea and 1.3 mL (9.3 mmol) of TEA in DCM (5 mL) at 0° C. The resulting mixture was stirred at 0° C. for 1 h and 25 h more at room temperature. Usual work up followed by silica gel column chromatography, eluting with hexane:EtOAc (4:1) gave 22a as a white solid (830 mg, 81% yield); mp 221-223° C.; IR (nujol) v 3258, 3164, 1714, 1635, 1615 cm⁻¹; ¹H NMR (CDCl₃) δ 1.53 (s, 18H), 1.56 (s, 18H), 3.92 (s, 2H), 7.48 (d, 2H, J=8.5 Hz), 7.65 (d, 2H, J=8.5 Hz), 7.92 (s, 2H), 10.45 (br, 2H), 11.70 (br, 2H); ¹³C NMR (CDCl₃) δ 28.1, 28.2, 37.2, 79.6, 83.7, 118.9, 119.7, 120.9, 135.3, 138.1, 144.2, 153.3, 153.4, 163.6; MS (ESI⁺) m/z 681.3575 [M+H]⁺.

### Dihydrochloride salt of N-(7-guanidino-9H-fluoren-2-yl)-guanidine (22b): Method C

Light brown solid (95%); mp decomposes over 175° C.; ¹H NMR (D₂O) δ 3.70 (s, 2H), 7.17 (d, 2H, J=8.0 Hz), 7.32 (s, 2H), 7.70 (d, 2H, J=8.0 Hz); ¹³C NMR (D₂O) δ 35.8, 120.6, 121.6, 123.7, 132.3, 139.1, 144.8, 155.7; MS (ESI⁺) m/z 281.1416 [M+H]⁺.

### Dihydrochloride salt of 4,4′-diguanidino-1,2-diphenylethane (23b): Method C

Light brown solid (94%); mp decomposes over 225° C.; ¹H NMR (D₂O) δ 2.94 (s, 4H), 7.14 (d, 4H, J=8.8 Hz), 7.27 (d, 4H, J=8.8 Hz); ¹³C NMR (D₂O) δ 35.4, 125.4, 129.6, 131.3, 141.1, 155.8; MS (ESI⁺) m/z 297.1969 [M+H]⁺.

### Spectroscopic Data of the New Boc Protected Derivatives:

### 1-[1,3-di(tert-butoxycarbonyl)-2-imidazolidinylimino]-4-benzylbenzene (4a): Method B

IR (nujol) v 1759, 1718 cm⁻¹; ¹H NMR (CDCl₃) δ 1.30 (s, 18H), 3.81 (s, 4H), 3.90 (s, 2H), 6.93 (d, 2H, J=8.0 Hz), 7.07 (d, 2H, J=8.0 Hz), 7.13-7.31 (m, 5H); ¹³C NMR (CDCl₃) δ 27.8, 41.4, 43.0, 82.6, 121.4, 125.7, 128.1, 128.7, 129.0, 135.2, 139.0, 141.7, 146.2, 150.2.

### 1-[1,3-di(tert-butoxycarbonyl)-2-imidazolidinylimino]-4-ethylbenzene (5a): Method B

IR (nujol) v 1760, 1723 cm⁻¹; ¹H NMR (CDCl₃) δ 1.06 (t, 3H, J=7.5 Hz), 1.21 (s, 18H), 2.46 (q, 2H, J=7.5 Hz), 3.70 (s, 4H), 6.81 (d, 2H, J=8.0 Hz), 6.94 (d, 2H, J=8.0 Hz); ¹³C NMR (CDCl₃) δ 15.6, 27.3, 27.8, 42.6, 81.9, 120.8, 127.4, 137.9, 138.5, 145.5, 149.8; MS (ESI⁺) m/z 390.2403 [M+H]⁺.

### 1-[1,3-di(tert-butoxycarbonyl)-2-imidazolidinylimino]-4-methylbenzene (6a): Method B

IR (nujol) v 1760, 1723 cm⁻¹; ¹H NMR (CDCl₃) δ 1.29 (s, 18H), 2.24 (s, 3H), 3.78 (s, 4H), 6.86 (d, 2H, J=8.0 Hz), 6.99 (d, 2H, J=8.0 Hz); ¹³C NMR (CDCl₃) δ 20.6, 27.7, 42.9, 82.4, 121.2, 129.0, 131.7, 138.8; 145.5, 150.2; MS (ESI⁺) m/z 376.2230 [M+H]⁺.

### [1,3-di(tert-butoxycarbonyl)-2-imidazolidinylimino]benzene (7a): Method B

IR (nujol) v 1715, 1798 cm⁻¹; ¹H NMR (CDCl₃) δ 1.31 (s, 18H), 3.81 (s, 4H), 6.92-7.08 (m, 3H), 7.18-7.24 (m, 2H); ¹³C NMR (CDCl₃) δ 27.7, 43.0, 82.6, 121.4, 122.4, 128.5, 139.1, 148.2, 150.2; MS (ESI⁺) m/z 362.2084 [M+H]⁺.

### 1-[1,3-di(tert-butoxycarbonyl)-2-imidazolidinylimino]-3,4-dimethylbenzene (8a): Method B

IR (nujol) v 1745, 1709 cm⁻¹; ¹H NMR (CDCl₃) δ 1.32 (s, 18H), 2.19 (s, 6H), 3.82 (s, 4H), 6.75 (d, 1H, J=8.0 Hz), 6.80 (s, 1H), 6.98 (d, 1H, J=8.0 Hz); ¹³C NMR (CDCl₃) δ 18.8, 19.5, 27.6, 42.8, 82.3, 118.4, 122.7, 129.4, 130.2, 136.0, 138.3, 145.6, 150.2.

### 5-[1,3-di(tert-butoxycarbonyl)-2-imidazolidinylimino]indan (9a): Method B

IR (nujol) v 1742, 1705 cm⁻¹; ¹H NMR (CDCl₃) δ 1.20 (s, 18H), 1.86-1.97 (m, 2H), 2.66-2.75 (m, 4H), 3.69 (s, 4H), 6.66 (d, 1H, J=8.0 Hz), 6.74 (s, 1H), 6.94 (d, 1H, J=8.0 Hz); ¹³C NMR (CDCl₃) δ 25.3, 27.4, 31.7, 32.4, 42.5, 81.8, 116.6, 119.0, 123.6, 137.5, 138.0, 143.7, 145.9, 149.8; MS (ESI⁺) m/z 402.2396 [M+H]⁺.

### 2-(5,6,7,8-Tetrahydro-naphthalen-2-ylimino)-imidazolidine-1,3-dicarboxylic acid di-tert-butyl ester (10a): Method B

IR (nujol) v 1745, 1710 cm⁻¹; ¹H NMR (CDCl₃) δ 1.28 (s, 18H), 1.66-1.77 (m, 4H), 2.58-2.71 (m, 4H), 3.77 (s, 4H), 6.65 (s, 1H), 6.69 (d, 1H, J=8.0 Hz), 6.87 (d, 1H, J=8.0 Hz); ¹³C NMR (CDCl₃) δ 23.1, 23.427.6, 28.7, 29.3, 42.9, 82.4, 118.7, 121.6, 129.0, 131.1, 136.6, 138.3, 145.3, 150.3.

### 1-[2,3-di(tert-butoxycarbonyl)guanidino]-4-benzylbenzene (11a): Method A

IR (nujol) v 3280, 3153, 1708, 1640, 1605 cm⁻¹; ¹H NMR (CDCl₃) δ 1.56 (s, 9H), 1.58 (s, 9H), 3.98 (s, 2H), 7.14-7.42 (m, 7H), 7.57 (d, 2H, J=8.0 Hz), 10.34 (br, 1H), 11.74 (br, 1H); ¹³C NMR (CDCl₃) δ 27.9, 28.0, 41.2, 79.3, 83.4, 122.1, 125.9, 128.3, 128.8, 129.2, 134.7, 137.4, 140.9, 153.1, 153.4, 163.4.

### 1-[2,3-di(tert-butoxycarbonyl)guanidino]-4-ethylbenzene (12a): Method A

IR (nujol) v 3285, 3146, 1720, 1639, 1606 cm⁻¹; ¹H NMR (CDCl₃) δ 1.22 (t, 3H, J=7.5 Hz), 1.52 (s, 9H), 1.55 (s, 9H), 2.62 (q, 2H, J=7.5 Hz), 7.16 (d, 2H, J=8.0 Hz), 7.51 (d, 2H, J=8.0 Hz), 10.27 (br, 1H), 11.70 (br, 1H); ¹³C NMR (CDCl₃) δ 15.6, 27.9, 28.1, 28.2, 79.3, 83.4, 122.1, 128.1, 134.2, 140.7, 153.2, 153.4, 163.5; MS (ESI⁺) m/z 386.2078 [M+Na].

### 1-[2,3-di(tert-butoxycarbonyl)guanidino]-4-methylbenzene (13a): Method A

IR (nujol) v 3267, 3153, 1717, 1644, 1608 cm⁻¹; ¹H NMR (CDCl₃) δ 1.52 (s, 9H), 1.56 (s, 9H), 2.33 (s, 3H), 7.14 (d, 2H, J=8.0 Hz), 7.48 (d, 2H, J=8.0 Hz), 10.27 (br, 1H), 11.67 (br, 1H); ¹³C NMR (CDCl₃) δ 20.8, 28.0, 28.1, 79.4, 83.4, 122.1, 129.3, 134.0, 134.3, 153.2, 153.5, 163.5.

### 1-[2,3-di(tert-butoxycarbonyl)guanidino]-3,4-dimethylbenzene (15a): Method A

IR (nujol) v 3285, 3156, 1718, 1642, 1610 cm⁻¹; ¹H NMR (CDCl₃) δ 1.52 (s, 9H), 1.55 (s, 9H), 2.23 (s, 3H), 2.26 (s, 3H), 7.09 (d, 1H, J=8.0 Hz), 7.29 (s, 1H), 7.42 (d, 1H, J=8.0 Hz), 10.23 (br, 1H), 11.69 (br, 1H); ¹³C NMR (CDCl₃) δ 19.1, 19.8, 28.0, 28.1, 79.3, 83.4, 119.7, 123.2, 129.8, 133.0, 134.3, 136.8, 153.2, 153.4, 163.6; MS (ESI⁺)m/z 386.2072 [M+Na]⁺.

### 5-[2,3-di(tert-butoxycarbonyl)guanidino]indan (16a): Method A

IR (nujol) v 3258, 3155, 1720, 1644, 1610 cm⁻¹; ¹H NMR (CDCl₃) δ 1.52 (s, 9H), 1.55 (s, 9H), 2.01-2.12 (m, 2H), 2.84-3.00 (m, 4H), 7.15 (d, 1H, J=8.0 Hz), 7.28 (d, 1H, J=8.0 Hz), 7.50 (s, 1H), 10.27 (br, 1H), 11.72 (br, 1H); ¹³C NMR (CDCl₃) δ 25.5, 27.9, 28.0, 32.2, 32.8, 79.2, 83.3, 118.4, 120.3, 124.2, 134.6, 140.7, 144.7, 153.2, 153.5, 163.5.

### 6-[2,3-di(tert-butoxycarbonyl)guanidino]-1,2,3,4-tetrahydronaphtalene (17a): Method A

IR (nujol) v 3162, 3101, 1723, 1626, 1611 cm⁻¹; ¹H NMR (CDCl₃) δ 1.53 (s, 9H), 1.56 (s, 9H), 1.73-1.84 (m, 4H), 2.68-2.85 (m, 4H), 7.02 (d, 1H, J=8.0 Hz), 7.24 (s, 1H), 7.36 (d, 1H, J=8.0 Hz), 10.22 (br, 1H), 11.70 (br, 1H); ¹³C NMR (CDCl₃) δ 23.0, 23.1, 28.1, 28.8, 29.4, 79.3, 83.3, 119.8, 122.5, 129.3, 133.7, 133.8, 137.4, 153.2, 153.5, 163.6; MS (ESI⁺) m/z 412.2206 [M+Na]⁺.

### 2,6-Bis[1,3-di(tert-butoxycarbonyl)-2-imidazolidinylimino]-9,10-dihydroanthracene (18a): Method B

IR (nujol) v 1742, 1715 cm⁻¹; ¹H NMR (CDCl₃) δ 1.30 (s, 36H), 3.82 (s, 4H), 3.87 (s, 8H), 6.87 (d, 2H, J=8.0 Hz), 6.96 (s, 2H), 7.12 (d, 2H, J=8.0 Hz); ¹³C NMR (CDCl₃) δ 27.7, 35.3, 43.0, 82.6, 119.1, 120.3, 127.4, 130.5, 136.7, 138.7, 145.9, 150.3; MS (ESI⁺) m/z 747.4084 [M+H]⁺.

### 2,7-Bis[1,3-di(tert-butoxycarbonyl)-2-imidazolidinylimino]-9H-fluorene (19a): Method B

IR (nujol) v 1702, 1686 cm⁻¹; ¹H NMR (CDCl₃) δ 1.27 (s, 36H), 3.72 (s, 2H), 3.84 (s, 8H), 6.98 (d, 2H, J=8.5 Hz), 7.11 (s, 2H), 7.54 (d, 2H, J=8.5 Hz); ¹³CNMR (CDCl₃) δ 27.7, 36.7, 43.0, 82.6, 117.7, 119.2, 120.4, 136.7, 138.6, 143.5, 146.4, 150.3; MS (ESI⁺) m/z 733.3889 [M+H]⁺.

### 4,4′-Bis[1,3-di(tert-butoxycarbonyl)-2-imidazolidinylimino]-1,2-diphenylethane (20a): Method B

IR (nujol) v 1719, 1691 cm⁻¹; ¹H NMR (CDCl₃) δ 1.32 (s, 36H), 2.75 (s, 4H), 3.82 (s, 8H), 6.92 (d, 4H, J=7.0 Hz), 7.06 (d, 4H, J=7.0 Hz); ¹³C NMR (CDCl₃) δ 27.8, 37.9, 43.0, 82.6, 121.4, 128.5, 136.3, 138.9, 146.0, 150.3; MS (ESI⁺) m/z 771.4027 [M+Na]⁺.

### 2,6-Bis[2,3-di(tert-butoxycarbonyl)guanidino]-9,10-dihydroanthracene (21a): Method A

IR (nujol) v 3265, 3159, 1712, 1643, 1618 cm⁻¹; ¹H NMR (CDCl₃) δ 1.53 (s, 18H), 1.56 (s, 18H), 3.91 (s, 4H), 7.24 (d, 2H, J=8.0 Hz), 7.43 (d, 2H, J=8.0 Hz), 7.55 (s, 2H), 10.34 (br, 2H), 11.70 (br, 2H); ¹³C NMR (CDCl₃) δ 28.1, 28.2, 35.6, 79.5, 83.6, 120.1, 121.1, 127.7, 132.9, 134.7, 137.1, 153.3, 153.5, 163.6; MS (ESI⁺) m/z 695.3768 [M+H]⁺.

### 4,4′-Bis[2,3-di(tert-butoxycarbonyl)guanidino]-1,2-diphenylethane (23a): Method A

IR (nujol) v 3290, 3157, 1716, 1647 cm⁻¹; ¹H NMR (CDCl₃) δ 1.51 (s, 18H), 1.53 (s, 18H), 2.84 (s, 4H), 7.10 (d, 4H, J=8.5 Hz), 7.50 (d, 4H, J=8.5 Hz), 10.28 (br, 2H), 11.68 (br, 2H); ¹³C NMR (CDCl₃) δ 27.8, 28.0, 37.1, 79.2, 83.3, 121.9, 128.6, 134.4, 137.9, 153.1, 153.2, 163.4; MS (ESI⁺) m/z 697.4073 [M+H]⁺.

Table of the combustion analysis data for the new target compounds:

| | | C | | H | | N | |
|---|---|---|---|---|---|---|---|
| Compd. | Formula | Calcd. | Found | Calcd. | Found | Calcd. | Found |
| 4b | C₁₆H₁₈ClN₃•1.4H₂O | 61.40 | 61.49 | 6.70 | 6.53 | 13.42 | 13.65 |
| 8b | C₁₁H₁₆ClN₃•0.2H₂O | 57.61 | 57.76 | 7.21 | 6.99 | 18.32 | 18.26 |
| 10b | C₁₁H₁₆ClN₃•1.3H₂O | 56.74 | 56.74 | 7.55 | 7.19 | 15.27 | 15.21 |
| 17b | C₁₁H₁₆ClN₃•0.8H₂O | 55.02 | 54.87 | 7.39 | 7.05 | 17.50 | 17.14 |
| 18b | C₂₀H₂₄Cl₂N₆•2.0H₂O | 52.75 | 52.68 | 6.20 | 5.94 | 18.45 | 18.22 |
| 19b | C₁₉H₂₂Cl₂N₆•1.8H₂O | 52.13 | 52.47 | 5.89 | 5.57 | 19.20 | 18.95 |
| 20b | C₂₀H₂₆Cl₂N₆•1.3H₂O | 54.01 | 53.81 | 6.48 | 6.14 | 18.89 | 18.73 |
| 21b | C₁₆H₂₀Cl₂N₆•2.3H₂O | 47.02 | 47.12 | 6.07 | 5.74 | 20.56 | 20.23 |

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

### REFERENCES

1. http://www.who.int/mental_health/management/depression/definition/en/2.
2. Elhwuegi, A. S. Central monoamines and their role in major depression. Prog. Neuropsychopharmacol. Biol. Psychiatry 2004, 28, 435-451.
3. (a) Callado, L. F.; Meana, J. J.; Grijalba, B.; Pazos, A.; Sastre, M.; Garcia-Sevilla, J. A. Selective increase of alpha2a-adrenoceptor agonist binding sites in brains of depressed suicide victims. J. Neurochem. 1998, 70, 1114-1123. (b) Gonzalez-Maeso, J.; Rodriguez-Puertas, R.; Meana, J. J.; Garcia-Sevilla, J. A.; Guimon, J. Neurotransmitter receptor-mediated activation of G-proteins in brains of suicide victims with mood disorders: Selective supersensitivity of alpha2A-adrenoceptors. Mol. Psychiatry 2002, 7, 755-767.
4. Mateo, Y.; Fernandez-Pastor, B.; Meana, J. J. Acute and chronic effects of desipramine and clorgyline on alpha2-adrenoceptors regulating noradrenergic transmission in the rat brain: A dual-probe microdialysis study. Br. J. Pharmacol. 2001, 133, 1362-1370.
5. Fernandez-Pastor, B.; Meana, J. J. In vivo tonic modulation of the noradrenaline release in the rat cortex by locus coeruleus somatodendritic alpha2-adrenoceptors. Eur. J. Pharmacol. 2002, 442, 225-229.
6. Devoto, P.; Flore, G.; Pani, L.; Gessa, G. L. Evidence for co-release of noradrenaline and dopamine from noradrenergic neurons in the cerebral cortex. Mol. Psychiatry 2001, 6, 657-664.
7. Mateo, Y.; Pineda, J.; Meana, J. J. Somatodendritic alpha2-adrenoceptors in the locus coeruleus are involved in the in vivo modulation of cortical noradrenaline release by the antidepressant desipramine. J. Neurochem. 1998, 71, 790-798.
8. Leonard, B. E. Neuropharmacology of antidepressants that modify central noradrenergic and serotonergic function: A short review. Hum. Psychopharm. Clin. 1999, 14, 75-81.
9. Rodriguez, F.; Rozas, I.; Ortega, J. E.; Meana, J. J.; Callado, L. F. Guanidine and 2-aminoimidazoline aromatic derivatives as alpha2-adrenoceptor antagonists, 1: Towards new antidepressants with heteroatomic linkers. J. Med. Chem. 2007, 50, 4516-4527.
10. Dardonville, C.; Rozas, I.; Meana, J.; Callado, K. I₂-imidazoline binding site affinity of a structurally different type of ligands. Bioorg. Med. Chem. 2002, 10, 1525-1533.
11. Grijalba, B.; Callado, L. F.; Meana, J. J.; Garcia-Sevilla, J. A.; Pazos, A. Alpha2-adrenoceptor subtypes in the human brain: A pharmacological delineation of [³H]RX-821002 binding to membranes and tissue sections. Eur. J. Pharmacol. 1996, 310, 83-93.
12. Fitzgerald, P. B.; Oxley, T. J.; Laird, A. R.; Kulkarni, J.; Egan, G. F.; Daskalakis, Z. J. An analysis of functional neuroimaging studies of dorsolateral prefrontal cortical activity in depression. Psychiatry Res. 2006, 148, 33-45.
13. Dardonville, C.; Goya, P.; Rozas, I.; Alsasua, A.; Martin, I.; Borrego, M. J. New aromatic iminoimidazolidine derivatives as alpha1-adrenoceptor antagonists: A novel synthetic approach and pharmacological activity. Bioorg. Med. Chem. 2000, 8, 1567-1577.
14. Kim, K. S.; Qian, L. Fire-retardant and intumescent compositions for cellulosic material. Tetrahedron Lett. 1993, 48, 7677-7680.
15. (a) Takimiya, K.; Yanagimoto, T.; Yamashiro, T.; Ogura, F.; Otsubo, T. Syntheses and properties of 11,11,12,12-tetracyano-2,6-anthraquinodimethane (TANT) and its 9,10-dichloro derivative as novel extensive electron acceptors. Bull. Chem. Soc. Jpn. 1998, 71, 1431-1435. (b) Yanagimoto, T.; Takimiya, K.; Otsubo, T.; Ogura, F. 11,11,12,12-Tetracyano-2,6-anthraquinodimethane (TANT) as a novel extensive electron acceptor. J. Chem. Soc., Chem. Commun. 1993, 6, 519-520.
16. Between others. (a) Berlinck, R. G. S.; Kossuga, M. H.; Nascimento, A. M. Guanidine derivatives. Science of Synthesis 2005, 18, 1077-1116. (b) Conyers, E.; Tye, H.; Whittaker, M. Preparation and evaluation of a polymer-supported Mukaiyama reagent. Tetrahedron Lett. 2004, 45, 3401-3404. (c) Powell, D. A.; Ramsden, P. D.; Batey, R. A. Phase-Transfer-Catalyzed alkylation of guanidines by alkyl halides under biphasic conditions: A convenient protocol for the synthesis of highly functionalized guanidines. J. Org. Chem. 2003, 68, 2300-2309. (d) Guisado, O.; Martinez, S.; Pastor, J. A novel, facile methodology for the synthesis of N,N′-bis(tert-butoxycarbonyl)-protected guanidines using polymer-supported carbodiimide. Tetrahedron Lett. 2002, 43, 7105-7109. (e) Musiol, H.-J.; Moroder, L. N,N′-Di-tert-butoxycarbonyl-1H-benzotnazole-1-carboxamidine derivatives are highly reactive guanidinylating reagents. Org. Lett. 2001, 3, 3859-3861. (f) Guo, Z.-X.; Cammidge, A. N.; Horwell, D. C. A convenient and versatile method for the synthesis of protected guanidines. Synthetic Commun. 2000, 30, 2933-2943. (g) Yong, Y. F.; Kowalski, J. A.; Thoen, J. C.; Lipton, M. A. A new reagent for solid and solution phase synthesis of protected guanidines from amines. Tetrahedron Lett. 1999, 40, 53-56. (h) Feichtinger, K.; Zapf, C.; Sings, H. L.; Goodman, M. Diprotected triflylguanidines: A new class of guanidinylation reagents. J. Org. Chem. 1998, 63, 3804-3805. (i) Miel, H.; Rault, S. Conversion of N,N′-bis(tert-butoxycarbonyl)guanidines to N-[N′-(tert-butoxycarbonyl)amidino]ureas. Tetrahedron Lett. 1998, 39, 1565-1568. (j) Yong, Y. F.; Kowalski, J. A.; Lipton, M. A. Facile and efficient guanylation of amines using thioureas and Mukaiyama's reagent. J. Org. Chem. 1997, 62, 1540-1542. (k) Drake, B.; Patek, M.; Lebl, M. A convenient preparation of monosubstituted N,N′-di(BOC)-protected guanidines. Synthesis 1994, 579-582. (I) Bernatowicz, M. S.; Wu, Y.; Matsueda, G. R. Urethane protected derivatives of 1-guanylpyrazole for the mild and efficient preparation of guanidines. Tetrahedron Lett. 1993, 34, 3389-3392.
17. (a) Arafa, R. K.; Brun, R.; Wenzler, T.; Tanious, F. A.; Wilson, W. D.; Stephens, C. E.; Boykin, D. W. Synthesis, DNA affinity, and antiprotozoal activity of fused ring dicationic compounds and their prodrugs. J. Med. Chem. 2005, 48, 5480-5488. (b) Boykin, D. W.; Tidwell, R. R.; Wilson, D. W.; Brun, R.; Arafa, R. K.; Stephens, C. E. Preparation of fused ring dicationic antiprotozoals and prodrugs thereof. PIXXD2 WO 2005051296 A2 20050609 CAN 143:43696 AN 2005:493466, 2005.
18. Balko, T. W. 2-(Substituted amino)-N-(3-substituted phenyl)-2-imidazoline-1-carbothioamides. USXXAM U.S. Pat. Nos. 4,195,092 1,9800,325 CAN 93:71781 AN 1980:471781 1980
19. Between others. (a) Katritzky, A. R.; Khashab, N. M.; Bobrov, S. The preparation of 1,2,3-trisubstituted guanidines. Helv. Chim. Acta 2005, 88, 1664-1675. (b) Genc, M.; Servi, S. Microwave-assisted synthesis of 2-(arylamino)-2-imidazolines on a solid support. Heteroat. Chem. 2005, 16, 142-147. (c) Xu, J.; Wang, Y.; Fu, J.; Zheng, W.; Zhang, H. Synthesis of imidazoline, oxazoline derivatives, and antihypertensive activity. Zhongguo Yaoke Daxue Xuebao 1998, 29, 336-341. (d) Randall, W. C.; Baldwin, J. J.; Cresson, E. L.; Tolman, R. L.; Weppelman, R. M.; Lyon, T. F. Multiple central alpha2-adrenoceptors of avian and mammalian species. Biochem. Pharmacol. 1983, 32, 1933-1940. (e) Merchan, F.; Garin, J.; Martinez, V.; Melendez, E. Synthesis of 2-aryliminoimidazolidines and 2-arylaminobenzimidazoles from methyl N-aryldithiocarbamates. Synthesis 1982, 482-484. (f) Trani, A.; Bellasio, E. Synthesis of 2-chloro-2-imidazoline and its reactivity with aromatic amines, phenols, and thiophenols. J. Heterocycl. Chem. 1974, 11, 257-262.
20. Between others. (a) Prisinzano, T.; Dukat, M.; Law, H.; Slassi, A.; MacLean, N.; DeLannoy, I.; Glennon, R. A. 2-(Anilino)imidazolines and 2-(benzyl)imidazoline derivatives as h5-HT_{1D} serotonin receptor ligands. Bioorg. Med. Chem. Lett. 2004, 14, 4697-4699. (b) Hirashima, A.; Rafaeli, A.; Gileadi, C.; Kuwano, E. Three-dimensional quantitative structure-activity studies of octopaminergic agonists responsible for the inhibition of sex-pheromone production in Hercoverpa armigera. Bioorg. Med. Chem. 1999, 7, 2621-2628. (c) Hirashima, A.; Pan, C.; Shinkai, K.; Tomita, J.; Kuwano, E.; Taniguchi, E.; Eto, M. Quantitative structure-activity studies of octopaminergic agonists and antagonists against nervous system of Locusta migratotia. Bioorg. Med. Chem. 1998, 6, 903-910.
21. Glennon, R. A.; Daoud, M. K.; Dukat, M.; Teitler, M.; Herrick-Davis, K.; Purohit, A.; Syed, H. Arylguanidine and arylbiguanide binding at 5-HT3 serotonin receptors: A QSAR study. Bioorg. Med. Chem. 2003, 11, 4449-4454.
22. Between others. (a) Paul, R.; Hallett, W. A.; Hanifin, J. W.; Reich, M. F.; Johnson, B. D.; Lenhard, R. H.; Dusza, J. P.; Kerwar, S. S.; Lin, Y. I.; Pickett, W. C.; Seifert, C. M.; Torley, L. W.; Tarrant, M. E.; Wrenn, S. Preparation of substituted N-phenyl-4-aryl-2-pyrimidinamines as mediator release inhibitors. J. Med. Chem. 1993, 36, 2716-2725. (b) Yang, H.; Henkin, J.; Kim, K. H.; Greer, J. Selective inhibition of urokinase by substituted phenylguanidines: Quantitative structure-activity relationship analyses. J. Med. Chem. 1990, 33, 2956-2961.
23. Between others. (a) Lefevre-Groboillot, D.; Boucher, J.-L.; Stuehr, D. J.; Mansuy, D.

Relationship between the structure of guanidines and N-hydroxyguanidines, their binding to inducible nitric oxide synthase (iNOS) and their iNOS-catalysed oxidation to NO. FEBS Journal 2005, 272, 3172-3183. (b) Bingham, A. H.; Davenport, R. J.; Gowers, L.; Knight, R. L.; Lowe, C.; Owen, D. A.; Parry, D. M.; Pitt, W. R. A novel series of potent and selective IKK2 inhibitors. Bioorg. Med. Chem. Lett. 2004, 14, 409-412. (c) Xian, M.; Fujiwara, N.; Wen, Z.; Cai, T.; Kazuma, S.; Janczuk, A. J.; Tang, X.; Telyatnikov, V. V.; Zhang, Y.; Chen, X.; Miyamoto, Y.; Taniguchi, N.; Wang, P. G. Novel substrates for nitric oxide synthases. Bioorg. Med. Chem. 2002, 10, 3049-3055. (d) Dukat, M.; Choi, Y.; Teitler, M.; Du Pre, A.; Herrick-Davis, K.; Smith, C.; Glennon, R. A. The binding of arylguanidines at 5-HT3 serotonin receptors: a structure-affinity investigation. Bioorg. Med. Chem. Lett. 2001, 11, 1599-1603. (e) Kreutzberger, A.; Gillessen, J. Antidiabetic hormones. III. 4,5,6-Trisubstituted 2-(4-toluidino)pyrimidine. J. Heterocycl. Chem. 1984, 21, 1639-1640. (f) Tilley, J. W.; Ramuz, H.; Levitan, P.; Blount, J. F. The synthesis of 3,5-diamino-1,2,4-oxadiazoles. Part 2.Helv. Chim. Acta 1980, 63, 841-859. (g) Srivastava, P. K.; Sharma, R. D.; Saleem, M. A convenient method for the preparation of monosubstituted guanidines. Current Science 1976, 45, 764-765.
24. Between others. (a) Vale-Silva, L. A.; Buchta, V.; Valentova, E. Effect of subinhibitory concentration of some established and experimental antifungal compounds on the germ tube formation in Candida albicans. Folia Microbiol. 2007, 52, 39-43. (b) Pham, T. A.; Jain, A. N. Parameter Estimation for scoring protein-ligand interactions using negative training data. J. Med. Chem. 2006, 49, 5856-5868. (c) Ganesh, V. K.; Muller, N.; Judge, K.; Luan, C.-H.; Padmanabhan, R.; Murthy, K. H. M. Identification and characterization of nonsubstrate based inhibitors of the essential dengue and West Nile virus proteases. Bioorg. Med. Chem. 2004, 13, 257-264. (d) Braunerova, G.; Buchta, V.; Silva, L.; Kunes, J.; Palat, K. Synthesis and in vitro antifungal activity of 4-substituted phenylguanidinium salts. Farmaco 2004, 59, 443-450. (e) Silva, F. P.; De-Simone, S. G. S1 subsite in snake venom thrombin-like enzymes: can S1 subsite lipophilicity be used to sort binding affinities of trypsin-like enzymes to small-molecule inhibitors? Bioorg. Med. Chem. 2004, 12, 2571-2587. (f) Rahman, A. A.; Daoud, M. K.; Dukat, M.; Herrick-Davis, K.; Purohit, A.; Teitler, M.; Taveres do Amaral, A.; Malvezzi, A.; Glennon, R. A. Conformationally-restricted analogues and partition coefficients of the 5-HT3 serotonin receptor ligands meta-chlorophenylbiguanide (mCPBG) and meta-chlorophenylguanidine (mCPG). Bioorg. Med. Chem. Lett. 2003, 13, 1119-1123. (g) Sturzebecher, J.; Schweinitz, A.; Schmalix, W. A.; Wikstrom, P. Synthetic urokinase inhibitors as potential anti-invasive drugs. IDrugs 2001, 4, 677-683. (h) Hajduk, P. J.; Boyd, S.; Nettesheim, D.; Nienaber, V.; Severin, J.; Smith, R.; Davidson, D.; Rockway, T.; Fesik, S. W. Identification of novel inhibitors of urokinase via NMR-based screening. J. Med. Chem. 2000, 43, 3862-3866. (i) Kuzmic, P.; Sideris, S.; Cregar, L. M.; Elrod, K. C.; Rice, K. D.; Janc, J. W. High-throughput screening of enzyme inhibitors: Automatic determination of tight-binding inhibition constants. Anal. Biochem. 2000, 281, 62-67. (j) Sperl, S.; Jacob, U.; De Prada, N. A.; Sturzebecher, J.; Wilhelm, O. G.; Bode, W.; Magdolen, V.; Huber, R.; Moroder, L. (4-aminomethyl)phenylguanidine derivatives as nonpeptidic highly selective inhibitors of human urokinase. Proc. Natl. Acad. Sci. U.S.A. 2000, 97, 5113-5118. (k) Nienaber, V.; Wang, J.; Davidson, D.; Henkin, J. Re-engineering of human urokinase provides a system for structure-based drug design at high resolution and reveals a novel structural subsite. J. Biol. Chem. 2000, 275, 7239-7248. (I) Magalhaes, A.; Monteiro, M. R.; Magalhaes, H. P. B.; Mares-Guia, M.; Rogana, E. Thrombin-like enzyme from Lachesis muta muta venom: Isolation and topographical analysis of its active site structure by means of the binding of amidines and guanidines as competitive inhibitors. Toxicon 1997, 35, 1549-1559. (m) Dukat, M.; Abdel-Rahman, A. A.; lsmaiel, A. M.; lngher, S.; Teitler, M.; Gyermek, L.; Glennon, R. A. Structure-activity relationships for the binding of arylpiperazines and arylbiguanides at 5-HT3 serotonin receptors. J. Med. Chem. 1996, 39, 4017-4026. (n) Min, K.-L.; Steghens, J.-P.; Henry, R.; Doutheau, A.; Collombel, C. Synthesis and differential properties of creatine analogs as inhibitors for human creatine kinase isoenzymes. Eur. J. Biochem. 1996, 238, 446-452.
25. Between others. (a) Blackburn, C.; Claiborne, C. F.; Cullis, C. A.; Dales, N. A.; Patane, M. A.; Stirling, M.; Stradella, O. G.; Weatherhead, G. S. Preparation of lactam compounds useful as protein kinase inhibitors. Patent PIXXD2 WO 2006041773 A2 20060420 CAN 144:412529 AN 2006:365250 2006. (b) Hughes, J. L.; Liu, Robert C. H. Pharmaceutical aromatic guanidine compositions. Patent USXXAM U.S. Pat. Nos. 3,908,013 1,975,0923 CAN 83:209416 AN 1975:609416 1975 (c) Clark, R. J.; Isaacs, A.; Walker, J. Derivatives of 3,4-xylidine and related compounds as inhibitors of influenza virus: Relationships between chemical structure and biological activity. Br. J. of Pharmacol. Chemother. 1958, 13, 424-435.
26. Rose, Y.; Ciblat, S.; Reddy, R.; Belley, A. C.; Dietrich, E.; Lehoux, D.; McKay, G. A.; Poirier, H.; Far, A. R.; Delorme, D. Novel non-nucleobase inhibitors of Staphylococcus aureus DNA polymerase IIIC. Bioorg. Med. Chem. Lett. 2006, 16, 891-896.
27. (a) Jerushalmy, Z.; Skoza, L.; Zucker, M. B.; Grant, R. Inhibition by guanidino compounds of platelet aggregation induced by adenosine diphosphate. Biochem. Pharmacol. 1966, 15, 1791-1803. (b) Ewins, A. J.; Barber, H. J.; Self, A. D. H. Diguanidine compounds. Patent GB 516289 19391229 CAN 35:37743 AN 1941:37743 1939.
28. Wong, W. C.; Jeon, Y. T.; Dhar, T. G. M.; Gluchowski, C. Imidazole and imidazoline derivatives as alpha2-adrenergic agonists. Patent PIXXD2 WO 9846572 A1 19981022 CAN 129:316226 AN 1998:706207, 1998.
29. Dardonville, C.; Rozas, I.; Alkorta, I. Similarity studies on guanidinium, imidazolinium, and imidazolium cations: Toward new bradykinin antagonists. J. Mol. Graph. Model. 1998, 16, 150-156.
30. Gonzalez-Maeso, J.; Rodriguez-Puertas, R.; Gabilondo, A. M.; Meana, J. J. Characterization of receptor-mediated [³⁵S]GTPγs binding to cortical membranes from postmortem human brain. Eur. J. Pharmacol. 2000, 390, 25-36.
31. Fillenz, M. In vivo neurochemical monitoring and the study of behaviour. Neurosci. Biobehay. Rev. 2005, 29, 949-962
32. Paxinos, G.; Watson, C. The rat brain in stereotaxic coordinates, 2^{nd} edition,

Academic Press: Orlando, 1986.
33. Author, A. B. U.S. Pat. No. 3,123,456, year.

1. A compound or a pharmaceutically acceptable salt thereof wherein the compound has the general formula (I) 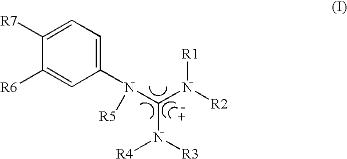
wherein the imine functional group can be at any one of the guanidine core carbon-nitrogen bonds; and
R₁ is H, N-tert-butoxycarbonate group, a lone pair of electrons or a C₁-C₅ alkyl chain which may be substituted or unsubstituted;
R₂ is H, a lone pair of electrons, a N-tert-butoxycarbonate group or a C₁-C₅ alkyl chain which may be substituted or unsubstituted;
R₃ is H, a lone pair of electrons, a N-tert-butoxycarbonate group or a C₁-C₅ alkyl chain which may be substituted or unsubstituted;
R₄ is H, N-tert-butoxycarbonate group or a lone pair of electrons or a C₁-C₅ alkyl chain which may be substituted or unsubstituted; or R₂ and R₃ together form a cyclic ring structure; and
R₅ is H, C₁-C₅ alkyl or a lone pair of electrons;
R₆ is H, an aryl, a C₁-C₅ alkyl aryl, phenylmethyl, 2-phenylethyl or a C₁-C₅ alkyl group, which may be substituted or unsubstituted, wherein when R₆ comprises phenylmethyl it is not substituted with a guanidine group or a 4,5-dihydro-1H-imidazoi-2-amine group; and
R₇ is H, an aryl, a C₁-C₅ alkyl aryl, phenylmethyl, 2-phenylethyl or a C₁-C₅ alkyl group, which may be substituted or unsubstituted, with the proviso that when R₇ comprises phenylmethyl it is not substituted with a guanidine group or a 4,5-dihydro-1H-imidazol-2-amine group; or
R₆ and R₇ together form part of a cyclic ring structure, a fused bicyclic or a fused tricyclic ring which can be unsubstituted or substituted,
with the proviso that when R₆ and R₇ form part of a fused bicyclic ring, R₆ and R₇ do not comprise a dioxane ring or a dioxolane ring, and
further provided that when R₂ and R₃ together form cyclic ring structure and when R₆ and R₇ form part of a fused bicyclic ring, R₆ and R₇ comprise an unsubstituted tetrahydronapthalene ring.

2. A compound according to claim 1 or a pharmaceutically acceptable salt thereof wherein R₆ and R₇ together form part of a fused tricyclic ring.

3. A compound according to claim 1 or a pharmaceutically acceptable salt thereof wherein the fused tricylic ring is a fluorene ring, or a dihydroanthracene ring which are unsubstituted or substituted at least one of a C₁-C₅ alkyl, an aryl, a C₁-C₅ alkyl aryl group, a guanidine group or a 4,5-dihydro-1H-imidazol-2-amine group.

4. A compound according to claim 1 or a pharmaceutically acceptable salt thereof wherein when R₆ or R₇ comprise 2-phenylethyl, or R₆ and R₇ together form part of a fused tricyclic ring the resulting structures are substituted with at least one of a guanidine group or a 4,5-dihydro-1H-imidazol-2-amine group.

5. (canceled)

6. A compound according to claim 1 or a pharmaceutically acceptable salt thereof selected from the group comprising

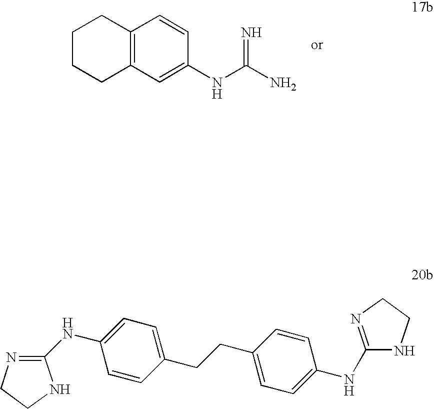

7. A compound according to claim 1 or a pharmaceutically acceptable salt thereof selected from the group comprising

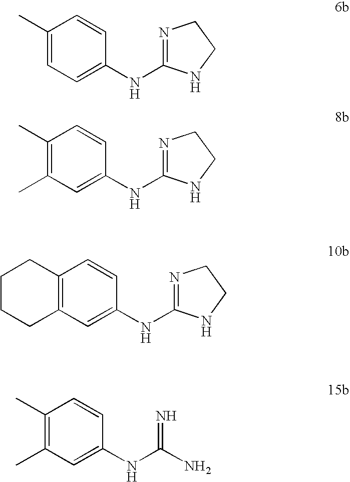

8. (canceled)

9. (canceled)

10. A pharmaceutical composition comprising a compound according claim 1, or a pharmaceutically acceptable salt thereof, together with a pharmaceutical acceptable carrier or excipient.

11. A pharmaceutical composition comprising a compound according to claim 6, or a pharmaceutically acceptable salt thereof, together with a pharmaceutical acceptable carrier or excipient.

12-24. (canceled)

25. A method of treating an alpha2-adrenoceptor associated disorder in a patient in need thereof, comprising administering to the patient a pharmaceutically effective amount of a compound according to claim 1 or a pharmaceutically acceptable salt thereof.

26. A method according to claim 25 wherein the compound is selected from the group comprising: 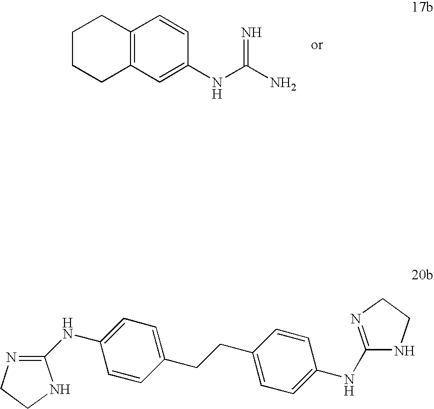
or a pharmaceutically acceptable salt thereof.

27. A method according to claim 25 wherein the compound is selected from the group comprising: 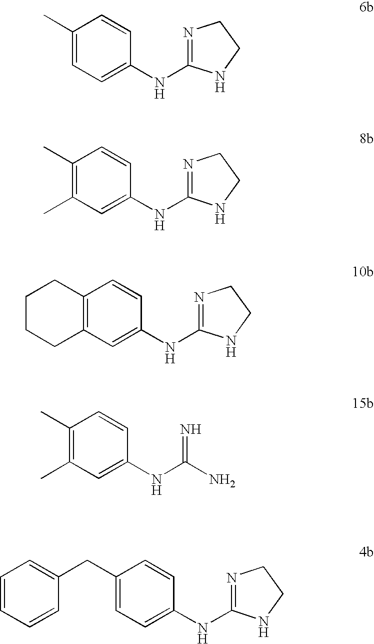
or a pharmaceutically acceptable salt thereof.

28-30. (canceled)

31. A method according to claim 26 wherein the alpha2-adrenoceptor associated disorder is a mental or neurological disorder.

32. A method according to claim 3| -w herein tile mental or neurological disorder is selected from at least one of depression or schizophrenia.

33. A method according to claim 32 wherein the mental or neurological disorder is depression.

34. A method according to claim 27 wherein the alpha2-adrenoceptor associated disorder is selected from at least one of analgesia, hypertension or glaucoma.

35. A method according to claim 33 wherein the compound is 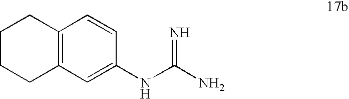
or a pharmaceutically acceptable salt thereof.
